(19) United States
(12) Patent Application Publication
(10) Pub. No.: US 2022/0010279 A1
(43) Pub. Date: Jan. 13, 2022

(54) KITS AND METHODS FOR PERFORMING OPTICAL DYNAMIC CLAMP ON EXCITABLE CELLS

(71) Applicants: CORNELL UNIVERSITY, Ithaca, NY (US); THE GEORGE WASHINGTON UNIVERSITY, Washington, DC (US)

(72) Inventors: Bonnie QUACH, Ithaca, NY (US); Trine KROGH-MADSEN, Ithaca, NY (US); David J. CHRISTINI, Ithaca, NY (US); Emilia ENTCHEVA, Washington, DC (US)

(21) Appl. No.: 17/282,181

(22) PCT Filed: Oct. 1, 2019
(86) PCT No.: PCT/US2019/053966

§ 371 Date: Apr. 1, 2021

Related U.S. Application Data
(60) Provisional application No. 62/739,558, filed on Oct. 1, 2018.

(51) Int. Cl. C12N 5/077; C12N 5/00; C12N 5/074

(52) U.S. Cl. CPC C12N 5/0657; C12N 5/0068; C12N 2529/00; C12N 2506/45; C12N 5/0696

(57) ABSTRACT

The present disclosure is directed to kits and methods for performing optical dynamic clamping on an excitable cell. In some embodiments, the excitable cell is selected from the group consisting of a neuron, a muscle cell and an excitable cell derived from an induced Pluripotent Stem Cell (IPSC). In a specific embodiment, the muscle cell is a cardiomyocyte.

### CROSS REFERENCE TO RELATED APPLICATION

This application claims the benefit of priority from U.S. Provisional Application No. 62/739,558, filed Oct. 1, 2018, the contents of which are incorporated herein by reference.

### STATEMENT REGARDING FEDERALLY SPONSORED RESEARCH OR DEVELOPMENT

This invention was made with government support under Fellowship No. F31HL134209-02 awarded by the National Institutes of Health. The government has certain rights in the invention.

### BACKGROUND

Optogenetic tools are typically used to generate a static current to stimulate action potentials or completely inhibit electrical activity. An optical action potential clamp has been used to uncover the dynamic contribution of Channelrhodopsin-2 (ChR2), a depolarizing opsin, during the cardiac action potential (Entcheva E, et al. Sci Rep. 2014, 4:srep05838; Williams J C, et al. PLOS Comput Biol. 2013, 9(9):e1003220). Several computational (Entcheva E, et al. Sci Rep. 2014, 4:srep05838; Karathanos T V, et al. Europace., 2014, 16 Suppl 4:iv69-iv76) and experimental studies (Govorunova E G, et al. Sci Rep, 2016, 6: 33530; Park S A, et al. Sci Rep. 2014, 4:6125) have used depolarizing and hyperpolarizing opsin to modulate the cardiac AP morphology. For example, activation of ChR2 by static light pulses delivered during different AP phases extended the action potential duration (APD) in neonatal rat ventricular myocytes (NRVM) (Park S A, et al. Sci Rep. 2014, 4:6125). Hyperpolarizing anion Channelrhodopsin 1 from Guillardia theta (GtACR1) was optically activated by static pulses to shorten the APD in NRVMs via forced hyperpolarization (Govorunova E G, et al. Sci Rep, 2016, 6: 33530). Static optogenetic manipulation can yield a range of AP responses depending on pulse timing, strength and duration (Entcheva E, et al. Sci Rep. 2014, 4:srep05838); however, it has inherent limitations when applied to multicellular tissue, where cells are at different phases of the AP at any given time. Computationally, a ChR2 model (Williams J C, et al. PLOS Comput Biol. 2013, 9(9):e1003220) was used to add a dynamic depolarizing current to simulate short QT syndrome and resemble a target AP, yet no real-time feedback was used (Karathanos T V, et al. Europace., 2014, 16 Suppl 4:iv69-iv76). Although this method worked well in silico, it would be critical to incorporate a real-time feedback loop to address inherent cell variability and make the approach AP-morphology-adaptive.

Archaerhodopsin TP009 (ArchT) has proven to be a useful tool to inhibit electrical activity in different excitable cells (Chow B Y, et al. Prog Brain Res. 2012, 196:49-61; Han X, et al. Front Syst Neurosci Front Syst Neurosci. 2011 Apr. 13; 5:18; Huff M L, et al. Proc Natl Acad Sci. 2013, 110(9):3597-602; Nussinovitch U, et al. Cardiovasc Res. 2014, 102(1):176-87; Li B, Yang X, et al. Brain Res. 2015, 1609:12-20; Tsunematsu T, et al. Optogenetics, Springer Japan; 2015, p. 249-63; Lux V, et al. Cereb Cortex. 2017, 27(1):841-51).

Cardiovascular toxicity is one of the major contributors to drug failure during clinical trials and drug withdrawal from the market (Redfern W S, et al. Cardiovasc Res. 2003, 58(1):32-45; Stevens J L, et al. Drug Discov Today. 2009, 14(3-4):162-7; Laverty H, et al. Br J Pharmacol. 2011, 163(4):675-93; Ferri N, et al. Pharmacol Ther. 2013, 138(3):470-84). Improving upon the sensitivity and specificity of pre-clinical cardiac toxicity assays would greatly mitigate the risk of drug-induced cardiotoxicity to patients and reduce failure of drugs during clinical trials. A limitation of these preclinical tests is that they rely on non-human models because human cardiac tissue is a very limited resource. Recent data in multiple areas, including cancer, neurodegenerative diseases and cardiovascular diseases indicate that animal models do not recapitulate physiology of human patients faithfully (Mak I W, et al. Am J Transl Res. 2014, 6(2):114-8; Olson H, et al. Regul Toxicol Pharmacol. 2000, 32(1):56-67; Scott S, et al. Amyotroph Lateral Scler Off Publ World Fed Neurol Res Group Mot Neuron Dis. 2008, 9(1):4-15). The development of human induced Pluripotent Stem Cell (iPSC)-derived cardiomyocytes (iPSC-CMs) offers a promising alternative to non-human models by providing a renewable source of human cardiomyocytes. Importantly, iPSC-CMs can be derived from a patient population of interest (Itzhaki I, et al. Nature. 2011, 471(7337):225-9; Guo L, et al. Toxicol Sci. 2013, 136(2):581-94; Harris K, et al. Toxicol Sci. 2013, 134(2):412-26; Sinnecker D, et al. Pharmacol Ther. 2014, 143(2):246-52; Dempsey G T, et al. J Pharmacol Toxicol Methods. 2016, 81:240-50).

Despite the advantages of human iPSC-CMs, concerns have been raised about their maturity, which may interfere with predicting the effect of drugs on adult human cardiac function. For example, a low or missing expression of the inward rectifier potassium current, I_{K1}, has been reported as the culprit for spontaneous beating in these cells, considered a sign of immaturity (Ma J, et al. Am J Physiol Heart Circ Physiol. 2011, 301(5):H2006-2017; Lieu D K, et al. Circ Arrhythm Electrophysiol. 2013, 6(1):191-201; Bett G C L, et al., Heart Rhythm, 10.12 (2013): 1903-1910). I_{K1} is critical for maintaining the resting membrane potential in adult cardiomyocytes and plays a role in late repolarization during an action potential. With insufficient I_{K1}, the resulting phenotype includes more depolarized triangular action potentials (APs) with longer action potential durations (APD). It has been demonstrated that electrically mimicking the low or missing I_{K1} in iPSC-CMs via dynamic clamp, a feedback-control based electrophysiological technique, can shift the electrophysiological phenotype to more adult-like (Bett G C L, et al., Heart Rhythm, 10.12 (2013): 1903-1910; Meijer van Putten R M E, et al. Front Physiol 6 (2015): 7; Verkerk A O, et al. Int J Mol Sci. 2017, 18(9)). Using this dynamic clamp approach can help in the study of pro- or anti-arrhythmic effects of drugs on cardiac electrical activity (Bett G C L, et al., Heart Rhythm, 10.12 (2013): 1903-1910). Unlike genetic overexpression of I_{K1}, the electronic expression of an ion channel via the dynamic clamp method can yield precise dosing and control. The user is able to electrically simulate the presence or absence of a desired current or to interface the patched cell with a mathematical model of another cell to simulate electrotonic interactions and cell behavior in the multicellular setting (Bett G C L, et al. Heart Rhythm. 2013, 10(12), 1903-1910; Meijer van Putten R M E, et al. Front Physiol., 2015; 6: 7; Ortega F A, et al. Methods Mol Biol. 2014, 1183:327-54; Brown T R, et al. Biophys J., 2016, 111(4):785-97; Devenyi R A, et al. J Physiol. 2017, 595(7):2301-17). Dynamic clamp provides high-content electrophysiology data, but its use is limited because of very low throughput and specialized technical expertise. The advent of automated patch clamp with multichannel capabilities addresses these barriers to use but requires more method development to make it compatible with cardiomyocytes from different species. Goversen et al. (2018) recently demonstrated using a Nanion Patchliner automated patch clamp device to inject IK1 via dynamic clamp into iPSC-CMs (Goversen B, et al. Front Physiol 8 (2018): 1094).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B. Description of EDC and ODC systems. Dynamic clamp is used to simulate the target current, I_{K1}, in iPSC-CMs. (A) The EDC system uses the electrode to measure the Vₘ and inject a current into the cell. (B) The ODC system utilizes the electrode to measure the Vₘ, but uses optical ArchT activation to inject the target current. Prior to implementing the ODC system, a calibration protocol is executed to obtain the parameters to generate a cell-specific ArchT model.

FIGS. 2A-2C. Calibration protocol to create a cell-specific ArchT model. (A) The calibration protocol consists of changing the light intensity and the holding potential to determine the ArchT model parameters, i.e. the light dependence (using the blue light-intensity ramp portion of the protocol) and voltage dependence (using the purple voltage-clamp steps in the protocol), for an individual cell. The bottom panel illustrates an example of the current output measured via patch clamp for one cell during the calibration protocol. (B and C) Current from the example trace in (A) during the light-intensity ramp (B) and voltage-clamp (C) steps, respectively. The currents were subtracted from the baseline, defined as an average of 10 msec prior to illumination.

FIGS. 3A-3C. Example demonstrating the results of the EDC and ODC platforms. Two stimulated APs from an example cell (cell 1, FIGS. 12A-12C and FIGS. 14A-14F) showing the effects of adding I_{target} (I_{K1}) while paced at three different frequencies: (A) 0.5 Hz, (B) 1 Hz, (C) 2 Hz. The gray, orange, and green traces represent the control without any current addition, adding I_{target} with EDC, and adding I_{target} with ODC, respectively. The top panels in each of (A) through (C) overlay the paced APs over time under control and both dynamic-clamp conditions, and the black triangles indicate when a stimulus current was delivered. In the middle panels, the traces represent the calculated target currents for EDC and ODC. The bottom panels show the calculated light intensity used to generate the target current. The time axis corresponds to the time within the full recording shown in FIGS. 12A-12C.

FIGS. 4A-4C. Example demonstrating the results of the EDC and ODC platforms. Figure is organized in the same manner as FIG. 3. This example is from cell 13 (FIGS. 12D-12F; and FIGS. 14A-14F). The time axis corresponds to the time within the full recording shown in FIGS. 12D-12F.

FIGS. 5A-5F. Summary of the effects of EDC or ODC on AP morphology. Pre-stimulation potential (A), fraction of repolarization (C) and triangulation (E) of individual cells at 2 Hz pacing in control (gray) and after adding an I_{K1} target current via EDC (orange) or ODC (green). The results of individual cells at 0.5 Hz and 1 Hz are displayed in FIGS. 14A-14F. Average of all cells and standard error mean (SEM) of the pre-stimulation potential (B), fraction of repolarization (D) and triangulation (E) by pacing frequency; No stimulated action potential could be measured for the control condition of cell 16 due to the high rate of spontaneous activity.

FIGS. 6A-6E. Example demonstrating the results of control, EDC and ODC after E-4031 addition. Example cell (cell 13, FIG. 7) showing the effects of adding I_{target} while paced at 0.5 Hz. The gray (A, D), orange (B, D, E) and green (C-E) traces represent the control without any current addition, adding I_{target} with EDC and adding I_{target} with ODC, respectively. (A-C) The darker traces represent the stimulated APs after E-4031 addition, while the light-colored traces represent the stimulated APs prior to E-4031 addition. (D) Overlays of AP traces from the three conditions after E-4031 addition. (E) Calculated target currents for EDC and ODC. Black triangles indicate when a stimulus current was delivered and provides a reference to which of the 10 paced APs in FIGS. 15A-15E are displayed. The time axis corresponds to the time within the full recording shown in FIGS. 15A-15E.

FIGS. 7A-7H. Summary of the effect of adding I_{K1} via EDC or ODC after E-4031 addition on AP morphology paced at 0.5 Hz. The APD₉₀ (A), pre-stimulation potential (C), fraction of repolarization (E) and triangulation (G) were measured without any current addition (grey), and with the addition of I_{target} via EDC (orange) or ODC (green) in individual cells (A) The empty markers represent the APD₉₀ prior to drug addition and the dark circles represent the APD₉₀ after E-4031 addition. Average of all cells and standard error mean (SEM) of the APD₉₀ after drug addition (B), pre-stimulation potential (D), fraction of repolarization (F) and triangulation (H) by pacing frequency.

FIGS. 8A-8D. Representative examples of calibration protocol outputs. The calibration protocol (A) can yield a variety of current outputs (B-D) that may be interrupted by repetitive large inward currents (C, D). These inward currents are associated with spontaneous contractions (C, D) which in some cases are suppressed with ArchT activation (D).

FIG. 9. Decrease in spontaneous activity with ODC. Average number of spontaneous events occurring during sequence of 10 stimulated APs at 0.5 Hz pacing. Each point represents a different cell and the lines connect the results from the same cell.

FIGS. 10A-10B. Difference in pre-stimulation potential between EDC and ADC does not correlate with differences in AP morphology characteristics between EDC and ADC. (A) Difference in pre-stimulation potential on difference in fraction of repolarization time. Results at 0.5 Hz, 1 Hz and 2 Hz pacing are purple, green, and red, respectively. (B) Difference in pre-stimulation potential on difference in triangulation. Results at 0.5 Hz, 1 Hz and 2 Hz pacing are purple, green, and red, respectively.

FIGS. 11A-11C. Amount of current decay during constant-intensity light pulses does not correlate with the difference in pre-stimulation potential between EDC and ADC. The amount of current decay was measured as an average difference between the initial current and the final current during the three constant-intensity (0.5 mW/mm²) light pulses during the calibration protocol. The amount of current decay was compared to the average difference in pre-stimulation potential (mV) between EDC and ADC at (A) 0.5 Hz, (B) 1 Hz, and (C) 2 Hz. Each point represents a different cell.

FIGS. 12A-12F. Entire trace of 10 paced APs demonstrating the results of the EDC and ODC platforms from example cells. Results from example cell 1 at (A) 0.5 Hz (B) 1 Hz and 2 Hz (C); and cell 10 at (D) 0.5 Hz (E) 1 Hz and (F) 2 Hz, showing the effects of adding I_{K1} while paced 10 times at 3 different frequencies as mentioned. The gray, orange and green traces represent the control without any current addition, adding I_{target} with EDC, and adding I_{target} with ODC, respectively. For each pacing rate, the top panel overlays the 10 paced AP traces over time under control and both dynamic-clamp conditions, and the black triangles indicate when a stimulus current was delivered. In the middle panel, the traces give the calculated target currents for EDC and ODC. The bottom panel shows the calculated light intensity used to generate the target current. The filled black triangles indicate when a stimulus current was delivered and provides a reference to which of the 10 paced APs in FIGS. 3A and 4B are displayed.

FIGS. 13A-13F. Representative example of a large undershoot after an AP and how EDC is able to compensate for the undershoot. Example cell (cell 3) showing the effects of adding IK1 while paced 10 times at 3 different frequencies: (A, B) 0.5 Hz, (C, D) 1 Hz, (E, F) 2 Hz. The gray, orange and green traces represent the control without any current addition, adding Itarget with EDC, and adding Itarget with ODC, respectively. The top panels overlay the 10 paced AP traces under control and both dynamic-clamp conditions, and the black triangles indicate when a stimulus current was delivered. In the middle panels, the traces give the calculated target currents for EDC and ODC. The bottom panels show the calculated light intensity used to generate the target current with ODC. The filled black triangles in the top panels (A, C, E) provide a reference for zoomed portions shown in B, D, and F, respectively.

FIGS. 14A-14F. Summary of the effects of EDC or ODC on AP morphology at different pacing frequencies. (A) Pre-stimulation potential at 0.5 Hz, (B) fraction of repolarization at 0.5 Hz, (C) triangulation at 0.5 Hz, (D) Pre-stimulation potential at 1 Hz, (E) fraction of repolarization at 1 Hz, (F) triangulation at 1 Hz of individual cells. Pacing in control (gray) and after adding an I_{K1} target current via EDC (orange) or ODC (green).

FIGS. 15A-15E. Entire trace of 10 paced APs demonstrating the results of the EDC and ODC platforms from example cell with E-4031 addition. Results from example cell 13 showing the effects of adding I_{K1} while paced 10 times at 0.5 Hz. The figure is organized in the same manner as FIGS. 12A-12F.

FIGS. 16A-16B. In silico model prediction shows that the error between RTXI and the amplifier has limited impact on dynamic clamp experiments. To predict the effects on dynamic clamp performance of the 5% error between the membrane potential measured by the amplifier versus the membrane potential reported by RTXI, an in silico approach was used, simulating I_{K1} dynamic clamp injection into an iPSC-CM computational model (Paci et al., 2013, Ann Biomed Eng., 2013 November; 41(11):2334-48) (“Paci”). The same equations from Paci were used for I_{K1} in the experiments and clamped intracellular [Na+] to mimic a patched cell. The dynamically clamped iPSC-CM model was run for 810 beats at a 1 Hz pacing rate with and without the amplifier calibration error and the last 10 APs were analyzed. (A) The top panel illustrates the last AP waveform with (dashed purple trace) and without (blue trace) the error. The bottom panel shows the corresponding target I_{K1}. The presence of the calibration error leads to an overestimation of this current during phase 4 of the AP, causing a small (about 2 mV) hyperpolarization of the resting membrane potential. (B) The resulting AP characteristics with and without the calibration error are very close, with about a 3% change in APD₉₀. Importantly, the predicted effect of the calibration error calculated here does not depend on how the dynamic clamp target current is added to the cell and is therefore expected to affect I_{target} calculated by the EDC and ODC systems equally.

FIGS. 17A-17B. In silico model predicting the effect of activation and deactivation kinetics on dynamic clamp performance and experimentally measured ArchT time constants. (A) To test that the kinetics of ArchT are not prohibitively slow for dynamic clamp, the ODC platform was stimulated using the Paci et al. (2013) iPSC-CM model with an added ArchT model having a single time constant for activation and deactivation. This time constant was varied to see how large of a delay could be tolerated. The ArchT current generated with different time constants are displayed in the panel above and sthe resulting stimulated APs are in the panel below. (B) Experimentally measured time constants of activation of ArchT in each cell.

FIGS. 18A-18B. Measuring stability of ArchT illumination during a voltage clamp and light clamp protocol. (A) The top panel shows the light clamp protocol and the bottom panel shows the voltage clamp protocol. This protocol was cycled through repeatedly to measure I_{ArchT} over time and at different holding potentials. (B) Results from a representative cell showing I_{ArchT} produced at different times of the protocol and at different holding potentials.

### SUMMARY OF THE DISCLOSURE

In one aspect, the present disclosure is directed to a kit comprising:

an excitable cell expressing at least one light-sensitive protein from an exogenous nucleic acid, wherein the light sensitive protein is selected from the group consisting of a light-sensitive ion channel and a light-sensitive ion pump; and

a computer readable media comprising instructions for performing an optical dynamic clamp on the cell, wherein the instructions comprise calculating a target ion current based on a measured membrane potential (Vm) using a predetermined relationship between a time-dependent Vm and an ion current; and calculating a target light intensity based on the target ion current, wherein exposing the excitable cell to the target light intensity results in an ion current from the light-sensitive protein that is equal to the target ion current.

In some embodiments, the at least one light sensitive protein is selective for an ion selected from the group consisting of a potassium ion, a sodium ion, a chloride ion, a H+ ion and a calcium ion.

In some embodiments, the excitable cell is selected from the group consisting of a neuron, a muscle cell and an excitable cell derived from an induced Pluripotent Stem Cell (iPSC). In a specific embodiment, the muscle cell is a cardiomyocyte. In a specific embodiment, the cardiomyocyte is an induced Pluripotent Stem Cell (iPSC)-derived cardiomyocyte.

In some embodiments, wherein the at least one light-sensitive protein is a channelrhodopsin, an anion-conducting channelrhodopsin, or a chimeric channelrhodopsin. In some embodiments, the channelrhodopsin is selected from the group consisting of Channelrhodopsin 1 (ChR1), Channelrhodopsin (ChR2), Volvox channelrhodopsin (VChR1), and Step function or bi-stable opsins (SFOs).

In some embodiments, the light-sensitive protein is selected from a halorhodopsin (NpHR), an enhanced halorhodopsin eNpHR2.0, an enhanced halorhodopsin eNpHR3.0, an archaerhodopsin (Arch), the fungal opsin Mac and an enhanced bacteriorhodopsin (eBR).

In some embodiments, the excitable cell expresses two different light-sensitive proteins, wherein each light sensitive protein is activated by a different wavelength of light. In some embodiments, one of the light sensitive proteins depolarizes the excitable cell when activated, and wherein the other light sensitive protein hyperpolarizes the excitable cell when activated.

Another aspect of the disclosure is directed to a method for modulating the electrophysiology of a cell comprising:

(i) providing an excitable cell, an electrode, and a light source with a controllable light intensity or a controllable light wavelength, wherein the excitable cell expresses at least one light-sensitive protein from an exogenous nucleic acid, and wherein the light-sensitive protein is selected from a light-sensitive ion channel or a light-sensitive ion pump;

(ii) forming a high resistance electrical seal between the electrode and a membrane of the cell;

(iii) measuring the membrane potential (Vm) of the cell with the electrode;

(iv) calculating a target ion current based on the measured Vm using a predetermined relationship between a time-dependent Vm and an ion current; and

(v) adjusting the light intensity or the light wavelength of the light source thereby controlling an ion current from the light-sensitive protein until the measured ion current is equal to the target ion current.

In some embodiments, the method further comprises repeating steps (iii) through (v). In some embodiments, the calculating and adjusting steps are carried out by a computer.

In some embodiments, the predetermined relationship is determined from a control excitable cell.

In some embodiments, step (iv) further comprises calculating a target light intensity based on the target ion current.

In some embodiments, the at least one light-sensitive protein is selective for an ion selected from the group consisting of a potassium ion, a sodium ion, a chloride ion, a H+ ion and a calcium ion.

In some embodiments, the excitable cell is selected from the group consisting of a neuron, a muscle cell and an excitable cell derived from an induced Pluripotent Stem Cell (iPSC). In a specific embodiment, the muscle cell is a cardiomyocyte. In a specific embodiment, the cardiomyocyte is an induced Pluripotent Stem Cell (iPSC)-derived cardiomyocyte.

In some embodiments, the at least one light-sensitive protein is a channelrhodopsin, an anion-conducting channelrhodopsin, or a chimeric channelrhodopsin. In some embodiments, the channelrhodopsin is selected from the group consisting of Channelrhodopsin 1 (ChR1), Channelrhodopsin (ChR2), Volvox channelrhodopsin 1 (VChR1), and Step function or bi-stable opsins (SFOs).

In some embodiments, the at least one light-sensitive protein is selected from a halorhodopsin (NpHR), an enhanced halorhodopsin eNpHR2.0, an enhanced halorhodopsin eNpHR3.0, an archaerhodopsin (Arch), the fungal opsin Mac and an enhanced bacteriorhodopsin (eBR).

In some embodiments, the excitable cell expresses at least two different light-sensitive proteins, wherein each light sensitive protein is activated by a different wavelength of light.

Another aspect of the disclosure is directed to a method for modulating the electrophysiology of a cell without using an electrode comprising:

(i) providing an excitable cell, a light source with a controllable light intensity or light wavelength, and an optical detector, wherein the excitable cell expresses at least one light-sensitive protein from an exogenous nucleic acid, and wherein the light-sensitive protein is selected from a light-sensitive ion channel or a light-sensitive ion pump, and wherein the excitable cell further expresses an optogenetic sensor expressed from an exogenous nucleic acid, and wherein the optical detector produces a signal indicating a membrane potential (Vm);

(ii) measuring the Vm of the cell by measuring a signal from optogenic sensor with the optical detector;

(iii) calculating a target ion current based on the measured Vm using a predetermined relationship between a time-dependent Vm and an ion current;

(iv) adjusting the light intensity or the light wavelength of the light source thereby controlling an ion current from the light-sensitive protein until the measured ion current is equal to the target ion current.

In some embodiments, the method comprises repeating steps (ii) through (iv).

In some embodiments, the calculating and adjusting steps are carried out by a computer. In some embodiments, the predetermined relationship is determined from a control excitable cell. In some embodiments, step (iii) further comprises calculating a target light intensity based on the target ion current.

In some embodiments, the light-sensitive protein is selective for an ion selected from the group consisting of a potassium ion, a sodium ion, a chloride ion, a H+ ion and a calcium ion.

In some embodiments, the excitable cell is selected from the group consisting of a neuron, a muscle cell and an excitable cell derived from an induced Pluripotent Stem Cell (iPSC).

In some embodiments, the muscle cell is a cardiomyocyte. In some embodiments, the cardiomyocyte is an induced Pluripotent Stem Cell (iPSC)-derived cardiomyocyte.

In some embodiments, the light-sensitive protein is a channelrhodopsin, an anion-conducting channelrhodopsin, or a chimeric channelrhodopsin. In a specific embodiment, the channelrhodopsin is selected from the group consisting of Channelrhodopsin 1 (ChR1), Channelrhodopsin (ChR2), Volvox channelrhodopsin 1 (VChR1), and Step function or bi-stable opsins (SFOs).

In some embodiments, the light-sensitive protein is selected from a halorhodopsin (NpHR), an enhanced halorhodopsin eNpHR2.0, an enhanced halorhodopsin eNpHR3.0, an archaerhodopsin (Arch), the fungal opsin Mac and an enhanced bacteriorhodopsin (eBR).

In some embodiments, the optogenetic sensor is selected from the group consisting of arc lightning, D3cpVenus, G-CaMP and ASAP1.

In some embodiments, the excitable cell expresses two different light-sensitive proteins, wherein each light sensitive protein is activated by a different wavelength of light.

### DETAILED DESCRIPTION

### Definitions

As used herein, the term “about” refers to an approximately ±10% variation from a given value.

As used herein “dynamic clamp” describes a method that detects an electrophysiological parameter (which may, for example, include current, voltage or capacitance) of a biological cell (or part thereof), and then applies a signal (for example, voltage or current) to the biological cell (or part thereof) to achieve a desired effect on the electrophysiological parameter. The step of applying the signal to the biological cell (or part thereof) requires the calculation of the amount of, for example, the voltage or current that must be applied to the cell (or part thereof) to produce the desired effect. Following the detection of an electrophysiological parameter and the subsequent application of the signal to the biological cell (or part thereof), the dynamic clamp continually repeats the process. See Prinz, A., Trends in Neurosciences, Volume 27, Issue 4, April 2004. Pages 218-24.

The dynamic clamp may comprise one or more electrodes. In one embodiment, the dynamic clamp comprises two electrodes which are in contact with a biological cell (or part thereof). In another embodiment, the dynamic clamp comprises one electrode which is in contact with a biological cell (or part thereof). These electrodes may provide a continuous clamp, a discontinuous clamp or a two electrode clamp. A continuous clamp comprises one electrode, and that electrode simultaneously and continuously detects an electrophysiological parameter and applies the signal (such as the voltage or current) to a cell (or part thereof). In contrast, a discontinuous clamp also comprises one electrode, but that electrode switches between detecting an electrophysiological parameter and applying the signal to the cell (or part thereof). In a two electrode clamp there are two electrodes: one electrode detects an electrophysiological parameter and the other applies the signal to the cell (or part thereof).

The dynamic clamp may also comprise a ground electrode. A ground electrode sets the ground reference point for electrophysiological measurements. The ground electrode may be in contact with a bath solution surrounding the biological cell (or part thereof). In one embodiment the ground electrode is a silver chloride coated silver wire. In another embodiment the ground electrode is a platinum electrode. The ground electrode may also be coated with agar.

As used herein, the term “voltage clamp” refers to a technique that allows an experimenter to “clamp” the cell potential at a chosen value. This makes it possible to measure how much ionic current crosses a cell's membrane at any given voltage. This is important because many of the ion channels in the membrane of a neuron are voltage-gated ion channels, which open only when the membrane voltage is within a certain range. Voltage clamp measurements of current are made possible by the near-simultaneous digital subtraction of transient capacitive and transmembrane currents that pass as the recording electrode and cell membrane are charged to alter the cell's potential. See Kandel E R et al., 2000, Principles of Neural Science, 4th ed., New York: McGraw-Hill. pp. 152-153.

As used herein, the term “current clamp” refers to a technique that records the membrane potential by injecting current into a cell through the recording electrode. Unlike in the voltage clamp mode, where the membrane potential is held at a level determined by the experimenter, in “current clamp” mode the membrane potential is free to vary, and the amplifier records whatever voltage the cell generates on its own or as a result of stimulation. This technique is used to study how a cell responds when electric current enters a cell. This is important for instance for understanding how neurons respond to neurotransmitters that act by opening membrane ion channels, which change a post-synaptic cells membrane potential. See Kandel E R et al., 2000, Principles of Neural Science, 4th ed., New York: McGraw-Hill. pp. 152-153.

As used herein, the term “waveform includes any variation (for example variations in the amplitude or frequency) in an electrophysiological parameter (for example the trans-membrane voltage) over time at a cell. Such variations may result from modulation of a number of ion channel or receptor types at the cell. In one embodiment, the waveform is an action potential or synaptic event. In a specific embodiment, the waveform is an action potential. A waveform at a biological cell (or part thereof) is generally produced by virtue of a functional inter-relationship between a number of different types of ion channels or receptors. Modulation of one, or a group of ion channels or receptors results in electrophysiological changes at the membrane of the cell, causing further ion channels to be modulated, resulting in a waveform. Ion channels including, for example, sodium channels, potassium channels, calcium channels, chloride channels and hyperpolarisation-activated cation channels may involved.

### General Description

### Excitable Cells

The present disclosure utilizes at least one excitable cell. As used herein, the term “excitable cell” refers to a cell that can be electrically excited and can generate an action potential.

In some embodiments, the excitable cell is selected from the group consisting of a neuron, a muscle cell, an excitable endocrine cell and an excitable cell derived from an induced Pluripotent Stem Cell (iPSC). In a specific embodiment, the excitable cell comprises a cardiomyocyte. In a specific embodiment, the excitable cell comprises a neuron. In a specific embodiment, the excitable endocrine cell comprises a pancreatic β cell.

In a specific embodiment, the excitable cell comprises an iPSC-derived cardiomyocyte. In a specific embodiment, the excitable cell comprises an iPSC-derived neuron.

### Light-Sensitive Proteins

The present disclosure utilizes at least one light sensitive protein. In some embodiments, the light sensitive protein is a light-sensitive ion channel or a light-sensitive ion pump. In some embodiments, the light sensitive protein is selective for an ion selected from the group consisting of a potassium ion, a sodium ion, a chloride ion, a H⁺ ion, and a calcium ion. As used herein, the term “selective for an ion” means that the light sensitive channel or pump specifically allows the transfer of a specific ion. For instance, a calcium-specific channel or a pump specifically transfers calcium ions and does not let other ions pass through.

In some embodiments, the light sensitive protein is permeable to the passage of more than one type of ion. In some embodiments, the light sensitive ion is permeable to ions showing a common charge. In a specific embodiment, the light sensitive ion is permeable to positively charged ions (cations). In a specific embodiment, the light sensitive ion is permeable to negatively charged ions (anions).

In some embodiments, the light-sensitive protein is a channelrhodopsin, an anion-conducting channelrhodopsin, or a chimeric channelrhodopsin.

As used herein, the term “channelrhodopsin” refers to a cation channel that depolarizes a cell upon light illumination. In some embodiments, the channelrhodopsin is activated by blue light. In some embodiments, the channelrhodopsin is activated by red light.

In a specific embodiment, the channelrhodopsin comprises Channelrhodopsin-1 (ChR1) that is proton (H⁺)-selective. In a specific embodiment, the channelrhodopsin comprises channelrhodopsin-2 (ChR2) which allows cations flow through non-specifically.

In a specific embodiment, the channelrhodopsin comprises Volvox-Channelrhodopsin-1 (VChR1) which is a red-shifted ChR variant. In some embodiments, VChR1 is activated by a light with a wavelength around 589 nm.

In some embosiments, the channelrhodopsin comprises a Step function or bi-stable opsin (SFO).

In a specific embodiment, the channelrhodopsin comprises a L132C mutation (CatCh) that increases the permeability for calcium and generates very large currents.

As used herein, the term “anion-conducting channelrhodopsin” refers to a light-gated ion channel that opens in response to light and lets negatively charged ions (such as a chloride ion) enter a cell.

As used herein, the term “chimeric channelrhodopsin” refers to a channelrhodopsin made by combining transmembrane helices from different channelrhodopsins, threby having a red spectral shift. In a specific embodiment, the chimeric channelrhodopsin comprises C1V1. In a specific embodiment, the chimeric channelrhodopsin comprises ReaChR.

In some embodiments, the light-sensitive protein is selected from a halorhodopsin (NpHR), an enhanced halorhodopsin eNpHR2.0, an enhanced halorhodopsin eNpHR3.0, an archaerhodopsin (Arch), the fungal opsin Mac and an enhanced bacteriorhodopsin (eBR).

### Light Sources

The present disclosure utilizes at least one light source. In some embodiments, the light from the light source has a fixed wavelength. In some embodiments, the light from the light source has an adjustable wavelength. In some embodiments, the light from the light source has a wavelength between 390 nm and 700 nm. In some embodiments, the light from the light source has a wavelength between 10 nm and 389 nm. In some embodiments, the light from the light source has a wavelength between 701 nm and 1 mm. In some embodiments, the light source emits laser light.

In some embodiments, the light source is a light-emitting diode (LED) light source. In some embodiments, the light source is a incandescent light source. In some embodiments, the light source is a fluorescent light source. In some embodiments, the light source is a halogen light source. In some embodiments, the light source is a high-intensity discharge lap (HID) light source. In some embodiments, the light source is a laser light source.

### Kits

An aspect of this disclosure is directed to a kit for performing optical dynamic clamping on an excitable cell. In some embodiments, the kit comprises an excitable cell expressing at least one light-sensitive protein from an exogenous nucleic acid, and a computer readable media comprising instructions for performing an optical dynamic clamp on the cell.

In some embodiments, the instructions for performing an optical dynamic clamp on the cell comprise calculating a target ion current based on a measured membrane potential (Vₘ) using a predetermined relationship between a time-dependent Vₘ and an ion current; and calculating a target light intensity based on the target ion current, wherein exposing the excitable cell to the target light intensity results in an ion current from the light-sensitive protein that is equal to the target ion current.

In some embodiments, the excitable cell exogenously expresses at least two light-sensitive proteins, and each light sensitive protein is activated by a different wavelength of light. In some embodiments, the excitable cell expresses exactly two light sensitive proteins that are activated by different wavelengths, and one of the light sensitive one of the light sensitive proteins depolarizes the excitable cell when activated, and wherein the other light sensitive protein hyperpolarizes the excitable cell when activated.

### Methods for Modulating the Electrophysiology of an Excitable Cell (Dynamic Clamping)

Another aspect of the disclosure is directed to a method for modulating the electrophysiology of a cell using optical clamping. In some embodiments, the method comprises:

(i) providing an excitable cell, an electrode, and a light source with a controllable light intensity or a controllable light wavelength, wherein the excitable cell expresses at least one light-sensitive protein from an exogenous nucleic acid, and wherein the light-sensitive protein is selected from a light-sensitive ion channel or a light-sensitive ion pump;

(ii) forming a high resistance electrical seal between the electrode and a membrane of the cell;

(iii) measuring the membrane potential (Vₘ) of the cell with the electrode;

(iv) calculating a target ion current based on the measured Vₘ using a predetermined relationship between a time-dependent Vₘ and an ion current; and

(v) adjusting the light intensity or the light wavelength of the light source thereby controlling an ion current from the light-sensitive protein until the measured ion current is equal to the target ion current.

In some embodiments, the steps (iii) through (v) are repeated to establish a dynamic clamp on the cellular electrophysiology. In some embodiments, the calculating and adjusting steps are carried out by a computer. In some embodiments, the predetermined relationship is determined from a control excitable cell.

In some embodiments, step (iv) further comprises calculating a target light intensity based on the target ion current.

In some embodiments, the excitable cell expresses at least two different light-sensitive proteins, wherein each light sensitive protein is activated by a different wavelength of light. In some embodiments, the excitable cell expresses exactly two light sensitive proteins that are activated by different wavelengths, and one of the light sensitive one of the light sensitive proteins depolarizes the excitable cell when activated, and wherein the other light sensitive protein hyperpolarizes the excitable cell when activated.

Another aspect of the disclosure is directed to a method for modulating the electrophysiology of a cell using contactless optical clamping (i.e., optical clamping without the use of an electrode). In some embodiments, the contactless method utilizes at least one optogenic sensor to monitor the membrane potential (Vₘ) or a target ion current. As used herein, the phrase “optogenic sensor” refers to a sensor that responds dynamically to changes in concentration of cellular molecules (e.g., concentration of ions) or changes in cellular action potential (voltage). In some embodiments, the optogenic sensor is fluorescent.

In some embodiments, the optogenic sensor is a voltage-responsive optogenic sensor. In a specific embodiment, the voltage-responsive optogenic sensor is arc lightning (Mancusso J J. et al., Exp Physiol. 2011 January; 96(1):26-3). In a specific embodiment, the voltage-responsive optogenic sensor is ASAP1 (Treger J S. et al., Elife. 2015 Nov. 24; 4:e10482).

In some embodiments, the optogenetic sensor is genetically encoded.

In some embodiments, the optogenetic sensor is a calcium sensor. In a specific embodiment, the optogenic sensor is a genetically-encoded calcium sensor. In a specific embodiment, the genetically-encoded calcium sensor is D3cpVenus (Tian et al., Nat Methods. 2009, (12):875-81) or G-CaMP (Nakai et al., Nat. Biotechnol. 2001, (2):137-41).

In some embodiments, the contactless optical clamping method comprises:

(i) providing an excitable cell, a light source with a controllable light intensity or light wavelength, and an optical detector, wherein the excitable cell expresses at least one light-sensitive protein from an exogenous nucleic acid, and wherein the light-sensitive protein is selected from a light-sensitive ion channel or a light-sensitive ion pump, and wherein the excitable cell further expresses an optogenetic sensor expressed from an exogenous nucleic acid, and wherein the optical detector produces a signal indicating a membrane potential (Vₘ);

(ii) measuring the Vₘ of the cell by measuring a signal from optogenic sensor with the optical detector;

(iii) calculating a target ion current based on the measured Vₘ using a predetermined relationship between a time-dependent Vₘ and an ion current;

(iv) adjusting the light intensity or the light wavelength of the light source thereby controlling an ion current from the light-sensitive protein until the measured ion current is equal to the target ion current.

In some embodiments, the steps (ii) through (iv) are repeated to establish a dynamic clamp on the cellular electrophysiology. In some embodiments, the calculating and adjusting steps are carried out by a computer. In some embodiments, the predetermined relationship is determined from a control excitable cell.

In some embodiments, step (iii) further comprises calculating a target light intensity based on the target ion current.

In some embodiments, the excitable cell expresses at least two different light-sensitive proteins, wherein each light sensitive protein is activated by a different wavelength of light. In some embodiments, the excitable cell expresses exactly two light sensitive proteins that are activated by different wavelengths, and one of the light sensitive one of the light sensitive proteins depolarizes the excitable cell when activated, and wherein the other light sensitive protein hyperpolarizes the excitable cell when activated.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Example 1: Materials and Methods

### Cell Culture

Cor.4U hiPSC-CMs (Axiogenesis, Cologne, Germany) were thawed, seeded, and maintained according to the protocols provided by the manufacturer. The cells were seeded on 0.5% gelatin-coated 8 mm coverslips and plated at 100,000 cells/mL. Cells were incubated for at least 7 days post thaw prior to use for experiments.

### Infection and Expression of ArchT

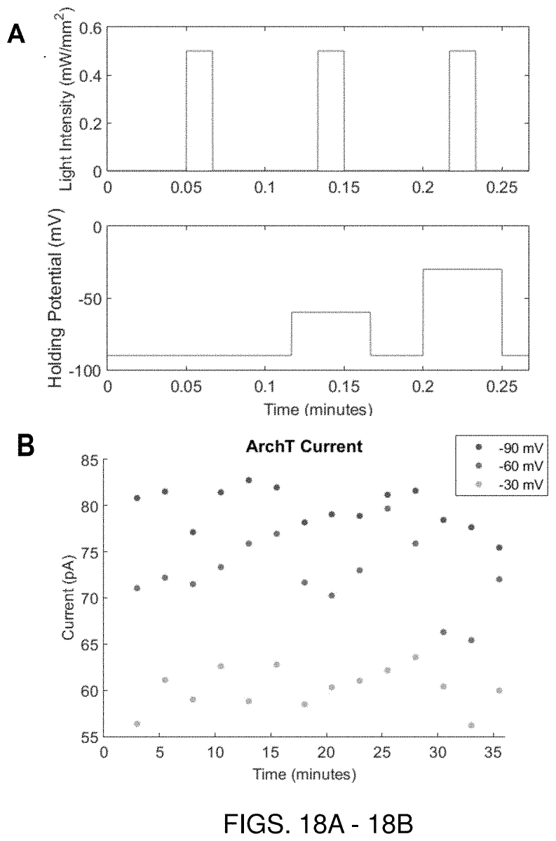

Adenoviral vector was constructed using the Addgene (Cambridge, Mass.) plasmid pAAV-CAG-ArchT-GFP, deposited by K. Deisseroth's laboratory (plasmid 20940) (Ambrosi C M, et al. Methods Mol Biol. 2014, 1181:215-28; Yu J, et al. Methods Mol Biol. 2016, 1408:303-17). ArchT was expressed in iPSC-CMs using MOIs of 250-300, as described in previously published protocols using an adenovirus (Ambrosi C M, et al. Methods Mol Biol. 2014, 1181:215-28; Yu J, et al. Methods Mol Biol. 2016, 1408:303-1). Determination of successful infection was confirmed via eGFP fluorescence. Functionality of ArchT was confirmed by illuminating the cells with an LED (M565L3, ThorLabs) at 595 nm through a 40× objective and observing the amount of hyperpolarization of the membrane potential under current clamp. Stability of I_{ArchT} was measured with a voltage and light clamp protocol over time to investigate rundown with ArchT illumination (FIGS. 18A-18B).

Electrophysiology

Borosilicate glass pipettes were pulled to a resistance of 1-3 MΩ using a flaming/brown micropipette puller (Model P-1000, Sutter Instrument). The pipettes were filled with intracellular solution containing (mM) 10 NaCl, 130 KCl, 1 MgCl₂, 10 CaCl₂, 5.5 Dextrose, 10 HEPES. For perforated patch, the pipette was first backfilled by dipping the pipette tip into the intracellular solution for 10 seconds. Only the very tip contained the intracellular solution without any gramicidin to minimize the amount of gramicidin exiting the pipette prior to obtaining a giga-ohm seal. The pipette was then filled with the intracellular solution containing 8 μg/mL gramicidin passed through a 0.25 μm filter. The pipette was filled about 60% with the intracellular solution containing 8 μg/mL gramicidin passed through a 0.25 μm filter. The high calcium concentration in the intracellular pipette solution serves to verify the integrity of the patch as patch rupture under these conditions would lead to immediate cell contracture (Ishihara K, et al. J Physiol. 2004, 540(3):831-41). The coverslips containing iPSC-CMs were placed in the bath and constantly perfused with an extracellular solution at 37° C. containing (mM) 137 NaCl, 5.4 KCl, 1 MgSO₄, 2 CaCl₂, 10 Dextrose, 10 HEPES. GFP-expressing single cells that were visibly contracting were chosen for experiments. Patch-clamp measurements were made by a patch-clamp amplifier (Model 2400, A-M Systems, Inc) controlled by the Real Time eXperiment Interface (RTXI) to coordinate the amplifier via the data acquisition card (PCI-6025E, National Instruments). The voltage was corrected for the calculated liquid junction potential of −2.8 mV. RTXI was also used to control the LED light intensity. The series resistance was less than 10 MΩ and was not compensated.

### Dynamic Clamp Experiments

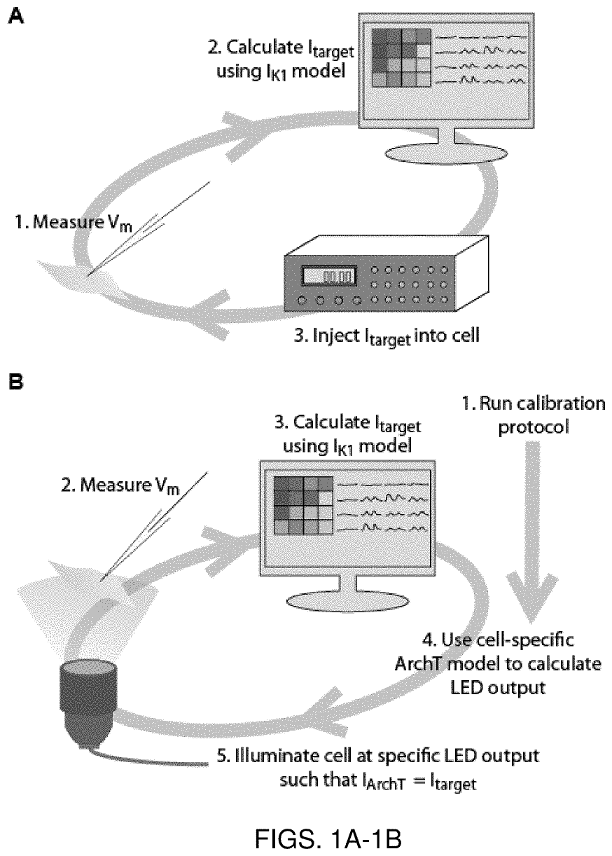

FIG. 1A depicts the schematic of the EDC system. At each time step, the electrode measures the membrane potential (Vₘ), which is then input into a mathematical model of I_{K1} to determine the amount of target current that should be generated at that measured Vₘ. The amplifier outputs the calculated target current in real-time, simulating the expression of an equivalent current within the cell.

FIG. 1B illustrates the ODC system. Similar to the EDC system, the membrane potential measured by an electrode is input into the mathematical model of the target current. The I_{K1} equations of the human ventricular myocyte model by ten Tusscher et al. (2004, Am. J. of Physiology, 286, H1573-H1589) were used. The maximum allowable I_{target} was set to 1 pA/pF because that was close to the maximum current that could be generated by ArchT in these cells. ArchT is a proton pump, generating a light- and voltage-sensitive outward current. There is no published validated mathematical model for the ArchT ion current. Instead, the inventors used an empirical equation that was tuned on a per-cell basis to generate the illumination Eₑ needed to achieve the target

$${I}_{ArchT} = \frac{{a}_{1} \cdot {V}_{m}^{*} + {a}_{2}}{{a}_{1} \cdot {V}_{rest} + {a}_{2}} \cdot {b}_{1} ( 1 - {e}^{- {b}_{2} \cdot Ee} ) , \qquad ( Eq . 1 )$$

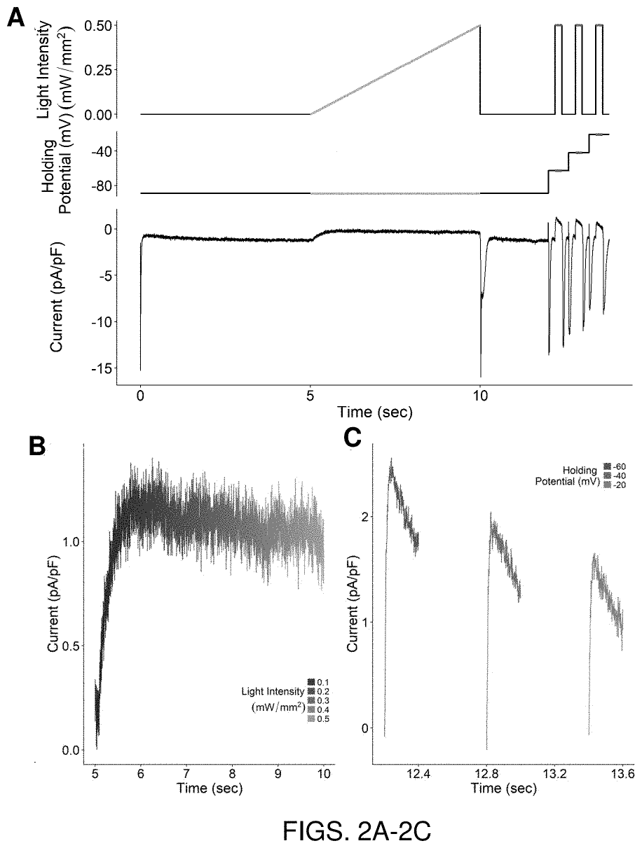

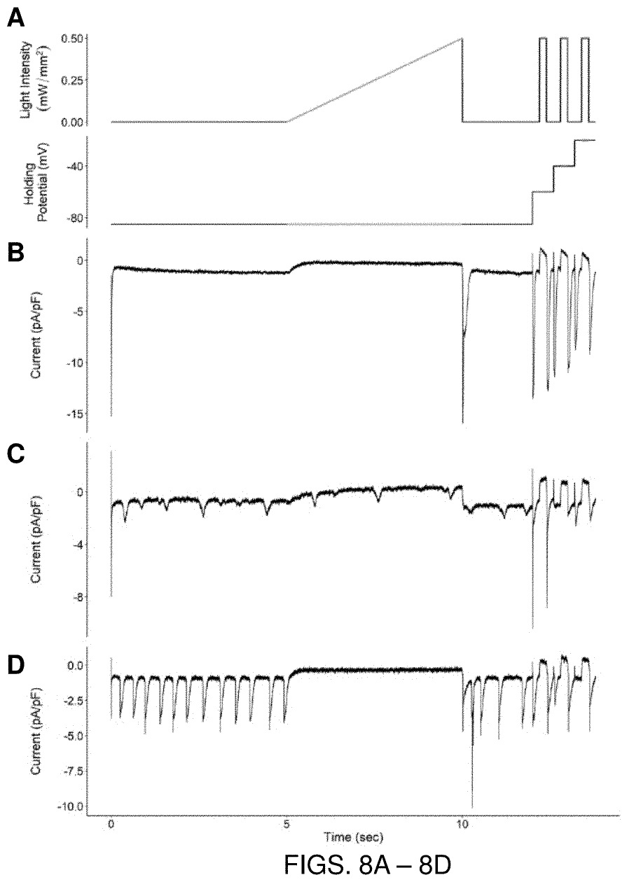

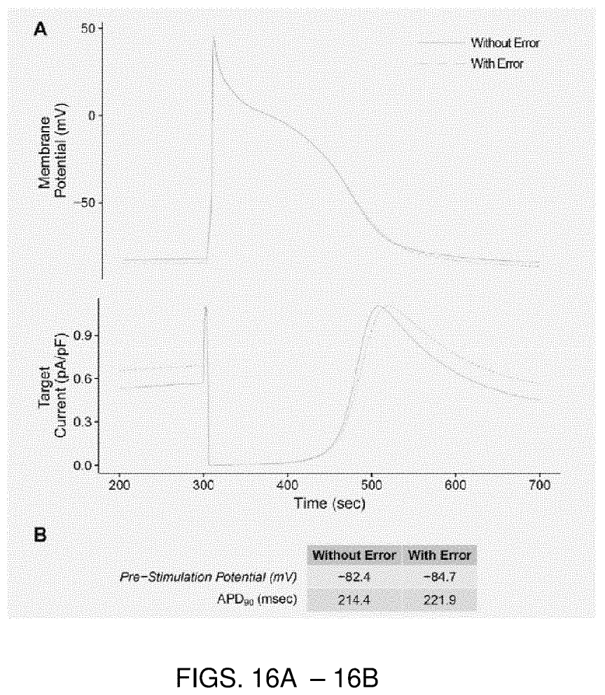

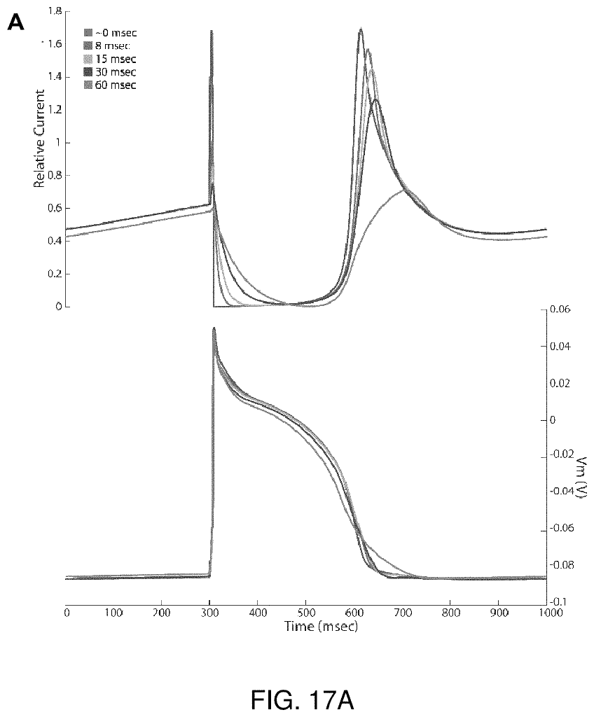

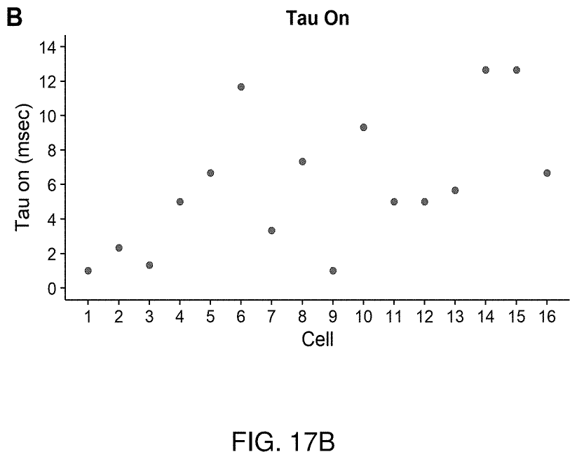

current, I_{ArchT}:
where the first component describes the voltage dependence, which is linearly affected by the membrane potential, and the second component describes the light intensity dependence of ArchT (Equation 1, FIG. 2). Due to an amplifier calibration error, the Vₘ used in this calculation by RTXI (Vₘ*) was later determined to be of 5% smaller amplitude than the real Vₘ as recorded by the amplifier. This error affects I_{ArchT} equally in the EDC and ODC systems and simulating its effects in a mathematical iPSC-CM model (Paci et al., 2013, Ann Biomed Eng., 2013 November; 41(11):2334-48) suggests that it has no significant impact on dynamic clamp performance (FIG. 16A). The parameter Vᵣₑᵥ is set to −85 mV, the reversal potential of potassium under experimental conditions. Eₑ represents the light intensity of the LED. a₁ and a₂ describe the cell-specific voltage dependence while b₁ and b₂ describe the cell-specific light intensity dependence. The values of these parameters are determined for each cell with a calibration protocol prior to running the ODC platform so that the ArchT model represents the characteristics from an individual cell. In about half the cells, inward current events are generated spontaneously even during voltage clamp at −85 mV (FIG. 8C, FIG. 8D). These spontaneous events may obscure the recorded current and thus the determination of the cell-specific ArchT parameters. However, cells in which these disturbances did occur were not associated with a reduction in ODC performance, measured as pre-stimulation potential, fraction of repolarization, and triangulation.

To test the feasibility of using ArchT to inject a target current, stimulated action potentials under the EDC system were compared to those of the ODC platform at three different pacing frequencies: 0.5, 1 and 2 Hz. The cells were stimulated 10 times at 0.5, 1 and 2 Hz sequentially under three conditions (the order of which was randomized): (A) control, in which no additional current was added, (B) addition of I_{target} with EDC, and (C) and addition of I_{target} with ODC.

### E4031 Addition

After the cells had undergone the aforementioned pacing protocol under the same three conditions, 500 nM E-4031 (a class III antiarrhythmic drug that blocks potassium channels, primarily of the human ether-a-go-go related gene (hERG) type) was perfused into the bath containing the coverslip of iPSC-CMs for 2 minutes. Experiments were again conducted under the same pacing protocol and conditions to measure the effect of I_{Kr} inhibition.

### Analysis

APDₓ was calculated by determining the time from stimulus to the time point at which the AP repolarized X % of the AP amplitude (AP peak—pre-stimulation potential). The AP peak was defined as the maximum membrane potential reached during the AP after delivered stimulus. The pre-stimulus potential is defined as an average of the membrane potential in the last 50 ms prior to delivering a stimulus current. The fraction of repolarization, calculated as (APD₉₀−APD₅₀)/APD₉₀, and triangulation, calculated as APD₉₀−APD₃₀, were used as metrics to quantify AP morphology. Data measured at a given pacing frequency in a cell were omitted from the analysis of AP characteristics if they contained more than one spontaneous event under EDC or ODC. This exclusion was necessary because spontaneous activity could affect the subsequent stimulated AP, obscuring the comparison between EDC and ODC.

### Example 2: Cell-Specific Calibration

Intrinsic cell-to-cell variability of ArchT expression and characteristics necessitated a calibration protocol that determines the cell specific parameters of the I_{ArchT} model (Eq. 1). The calibration protocol consists of a voltage-clamp protocol and a light-clamp protocol (FIG. 2A). The light intensity ramp of the protocol highlighted in blue is used to determine the parameters describing light dependence of I_{ArchT}, while the voltage steps highlighted in purple are used to obtain the parameters quantifying its voltage dependence. FIG. 2B depicts the example current trace during the light intensity ramp on an extended time axis and FIG. 2C shows the current during each of the three voltage clamp steps. These data are used to obtain the cell-specific parameters by determining the best-fit line using a nonlinear least squares analysis. By deriving the cell-specific ArchT parameters, the light intensity can be accurately calculated to activate ArchT and generate the target current in individual cells.

### Example 3: ODC Achieves Results Similar to EDC

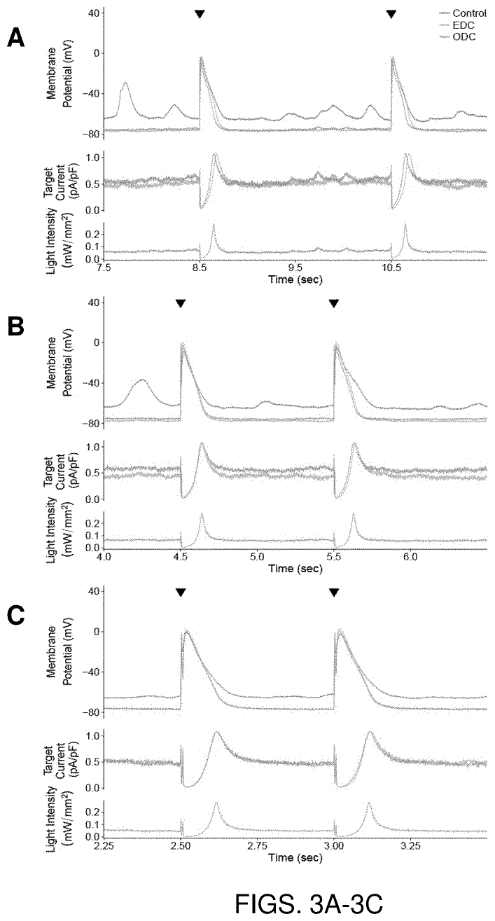

After obtaining the cell-specific parameters for Eq. 1, the ODC method was used and the performance of ODC was compared to EDC. Results from one representative cell paced at 0.5, 1 and 2 Hz are illustrated in FIG. 3. The control yielded a lot of spontaneous activity, making it difficult to trigger a stimulated AP or skewing the subsequent stimulated AP. The EDC and ODC platforms hyperpolarize the membrane potential and inhibit the occurrence of spontaneous events. The stimulated APs in the EDC and ODC conditions are very similar, demonstrating that the EDC and ODC platforms yield nearly identical stimulated APs at different pacing frequencies despite their fundamentally different methods of injecting a current. The EDC target current also overlaps with the ODC target current, as would be expected to generate similar APs. It is important to note that the ODC I_{target} is the calculated current, not the measured current. Similar to I_{K1}, the target current is on between APs to maintain the resting membrane potential. During the early phases of the action potential, the target current turns off and then increases during repolarization as I_{K1} would behave. These results indicate that the ODC platform is able to calculate a target current, determine the light intensity needed to generate that target current, adjust the LED output, activate the optical tool, and successfully generate the target current. In short, it demonstrates the feasibility of ArchT to inject a target current analogous to injection via an electrode.

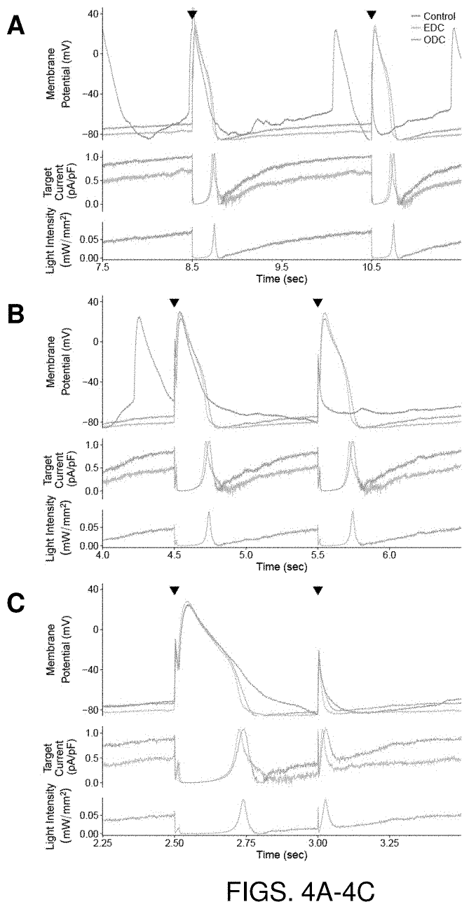

While the cell presented in FIG. 3 is representative of the most common ODC performance (11 of 16 cells with similar results), in a subset of cells (5 of 16), ODC was unable to maintain the resting membrane potential as well as EDC so that there is a greater than 5 mV difference in the pre-stimulation potential between EDC and ODC. FIG. 4 shows a cell that illustrates this behavior. The gradual depolarizing drift of the ODC membrane potential led to an increase in the target current (and therefore light intensity) as the ODC attempted to hyperpolarize the membrane potential. Although the light intensity did increase, the membrane potential could not be maintained, potentially because ArchT did not generate the required target current. Despite the difference in the pre-stimulation potential, both dynamic clamp systems had a similar effect on the overall morphology of the stimulated APs, which can be seen by the degree of overlap of the EDC and ODC AP traces. EDC and ODC both inhibited spontaneous activity similarly to the cell in FIG. 3, allowing for better measurement of the AP waveform whereas without outpacing the intrinsic spontaneous rate, it was difficult under the control condition to measure an AP without a preceding spontaneous event.

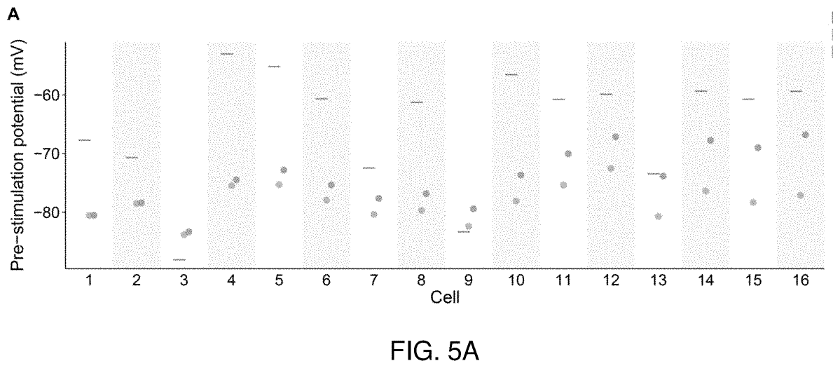

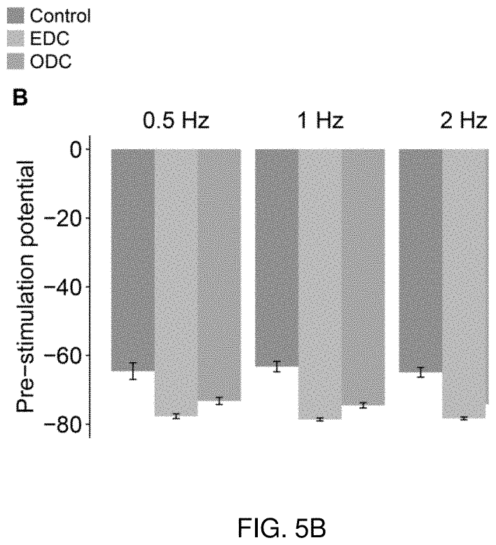

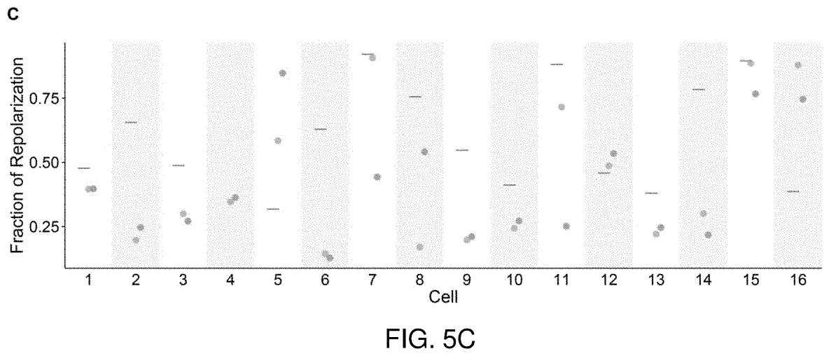

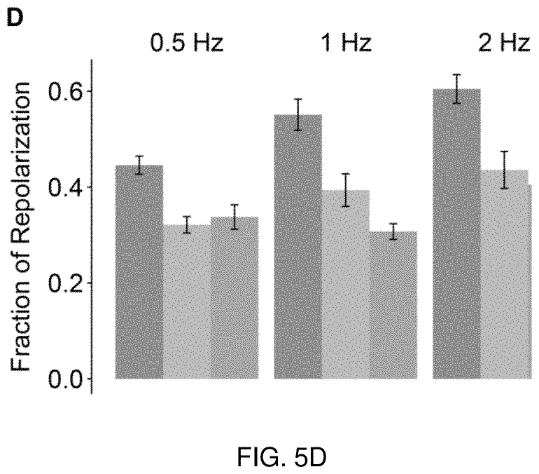

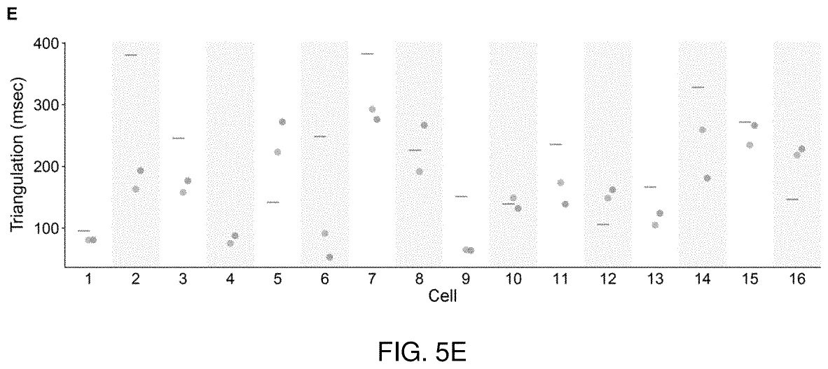

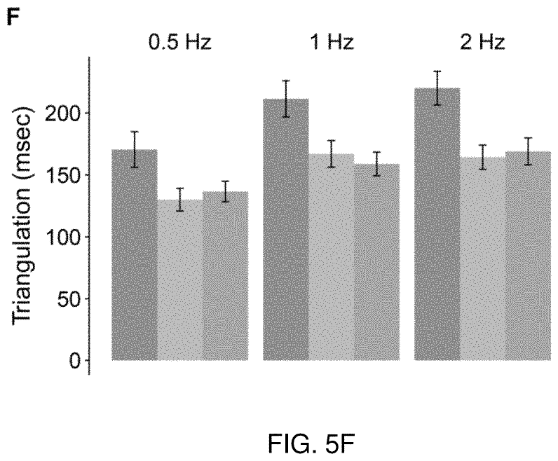

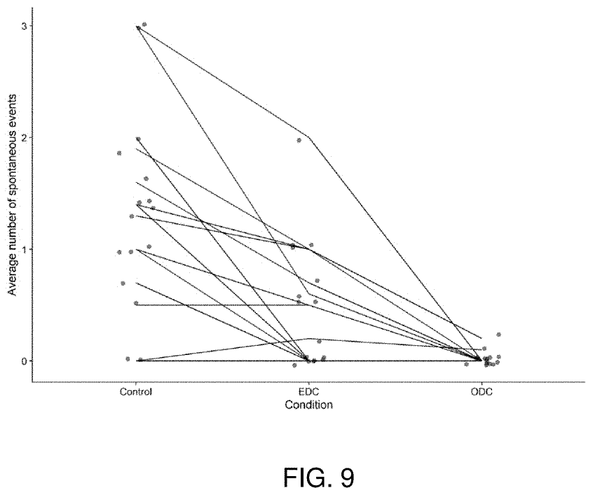

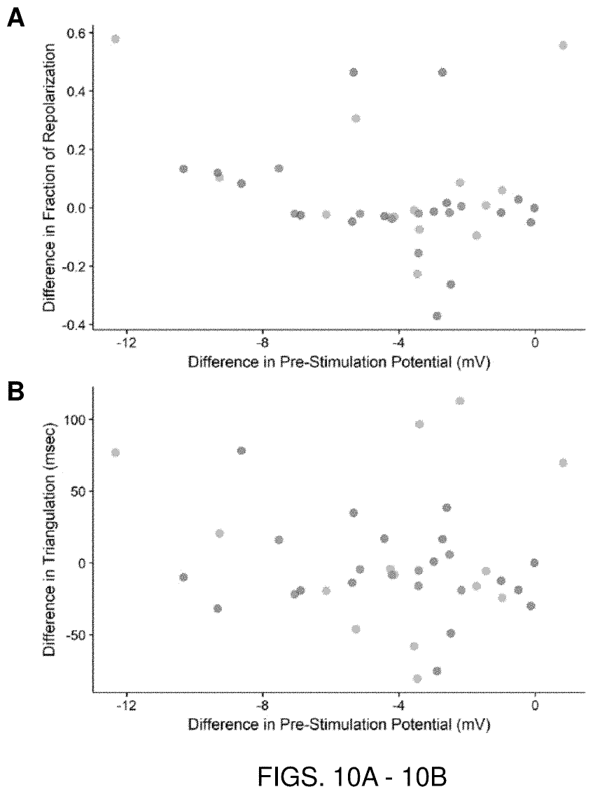

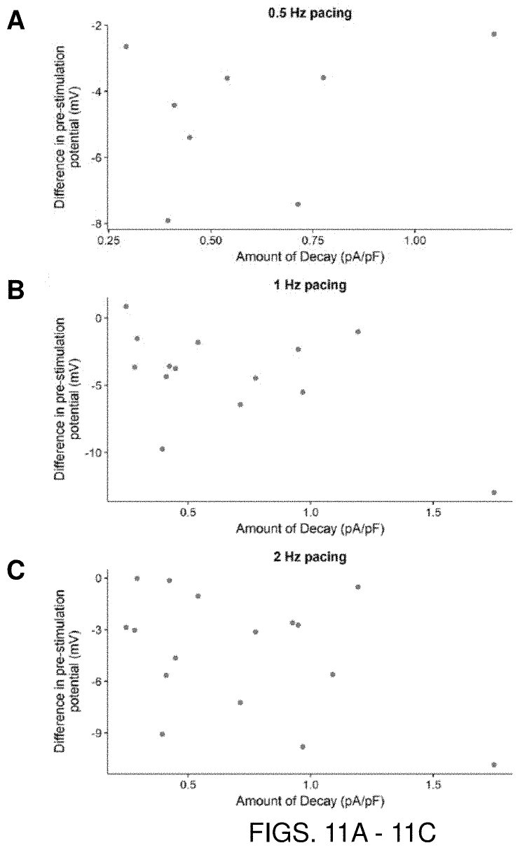

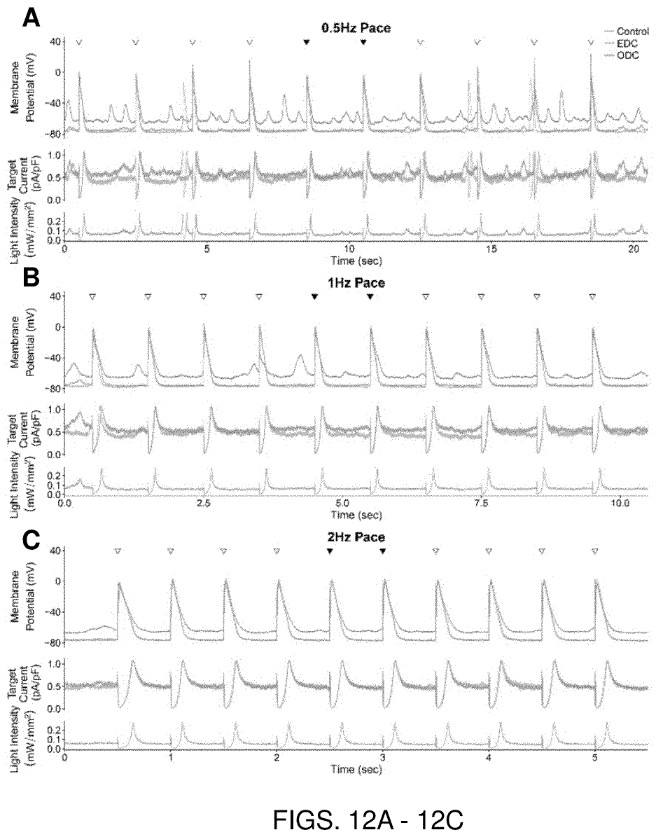

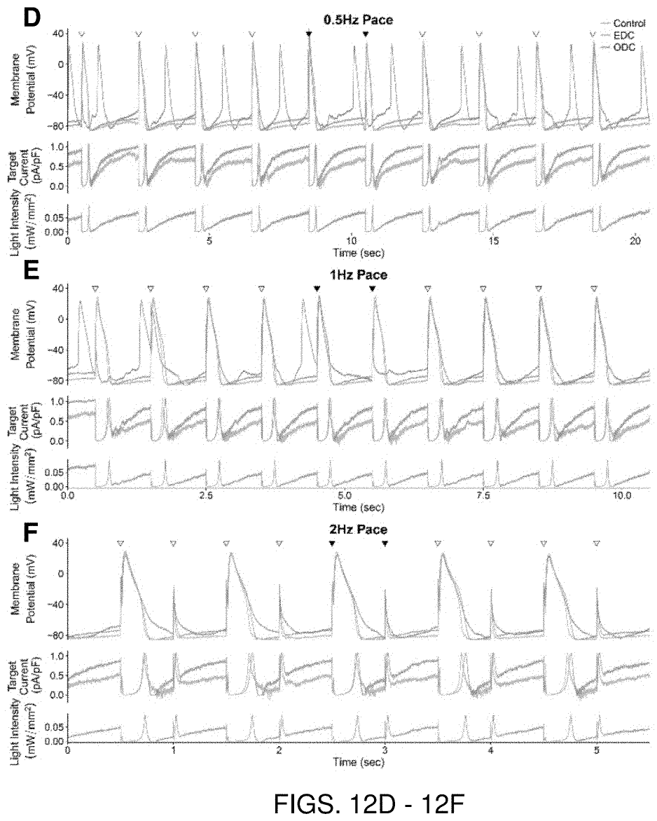

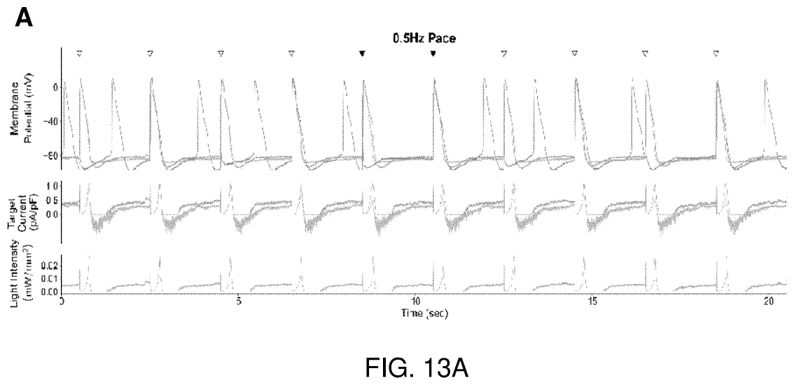

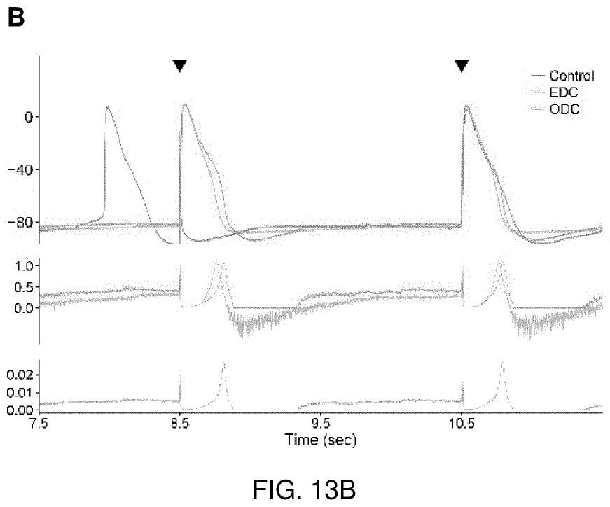

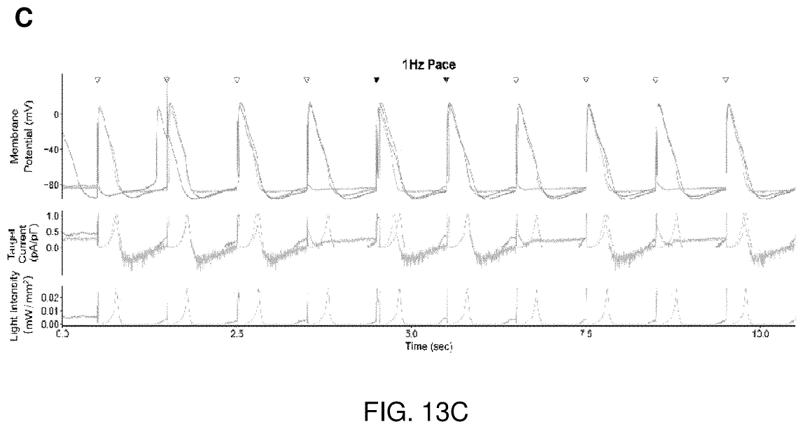

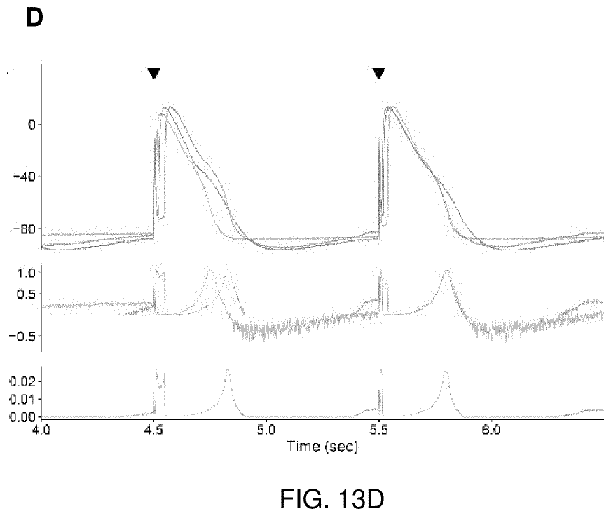

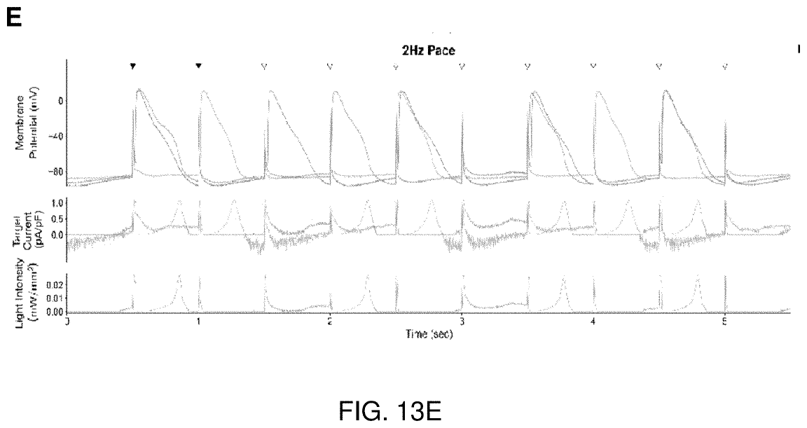

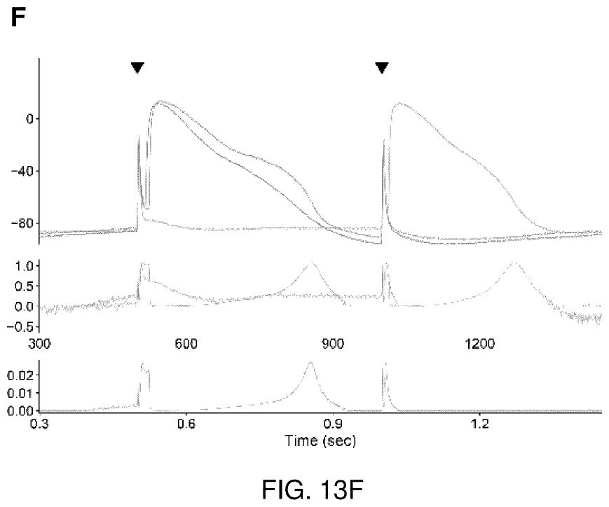

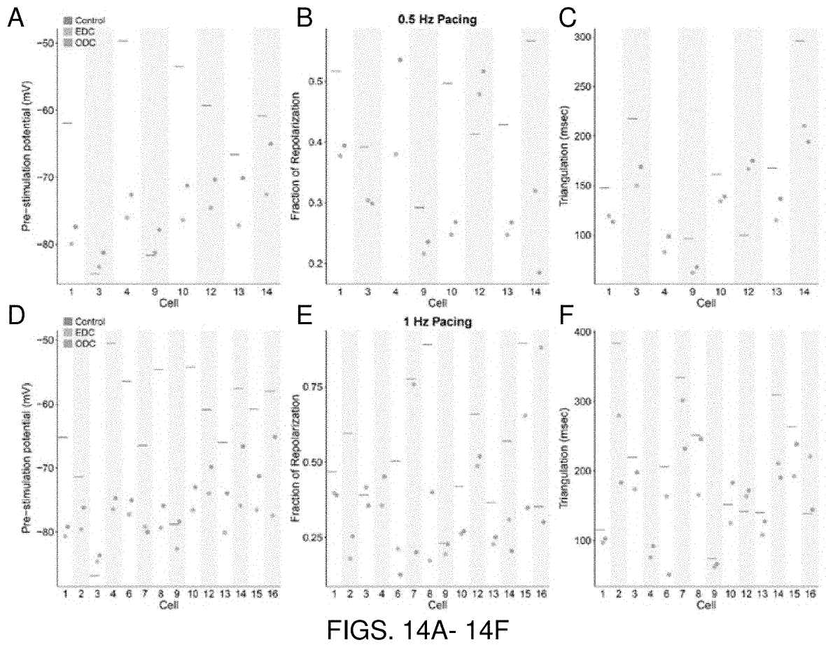

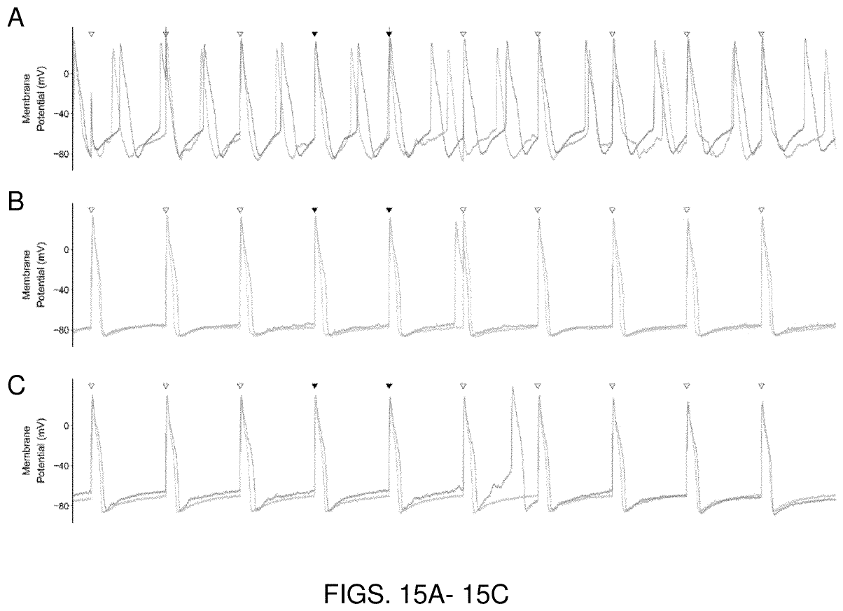

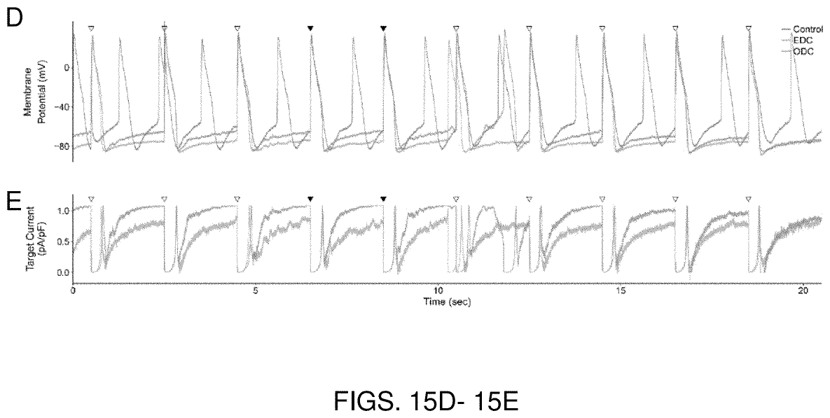

FIG. 5 summarizes the effect of EDC and ODC across 16 individual cells on the pre-stimulation potential (FIG. 5A), triangulation (FIG. 5B) and the fraction of repolarization (FIG. 5C). EDC and ODC have the advantage over the control condition of suppressing the rate of spontaneous activity, especially when the pacing rate is less than the intrinsic rate (FIG. 9). Both dynamic clamp systems hyperpolarize the pre-stimulation potential compared to the control (FIG. 5A). Ideally, the inventors would expect ODC to hyperpolarize the pre-stimulation potential to the same value as EDC. In 11 of 16 cells (e.g., cell in FIG. 3), the pre-stimulation potential in both ODC and EDC were within 5 mV of each other. However, as mentioned above, in 5 of 16 cases, (e.g., cell in FIG. 4), the pre-stimulation potential of the ODC was depolarized more than 5 mV relative to EDC because ODC did not maintain the membrane potential as well as EDC. On average, ODC did not hyperpolarize the pre-stimulation potential to the same magnitude as EDC (FIG. 5D). Despite the more depolarized pre-stimulation potential, ODC had similar effects as EDC on the overall AP morphology. As one marker of AP morphology fraction of repolarization, which quantifies the fraction of the AP that is spent in the repolarization phase, was used. The fraction of repolarization was expected to decrease with dynamic clamp, given that IK1 contributes to late repolarization. The ODC affected the fraction of repolarization by the same magnitude as EDC on average (FIG. 5D) and this is also seen on an individual basis (FIG. 5C). Triangulation provides another marker of the overall shape of the AP. In most cells, ODC altered triangulation by similar magnitudes as EDC (FIG. 5E). The overall average across all pacing frequencies also demonstrates that ODC had a similar effect on triangulation as EDC (FIG. 5F). In summary, comparing the AP characteristics under EDC and ODC reaffirms that ArchT is able to recapitulate similar effects as an electrode on AP morphology.

### Example 4: ODC Platform Detects Effect of I_{Kr} Inhibition Similar to EDC

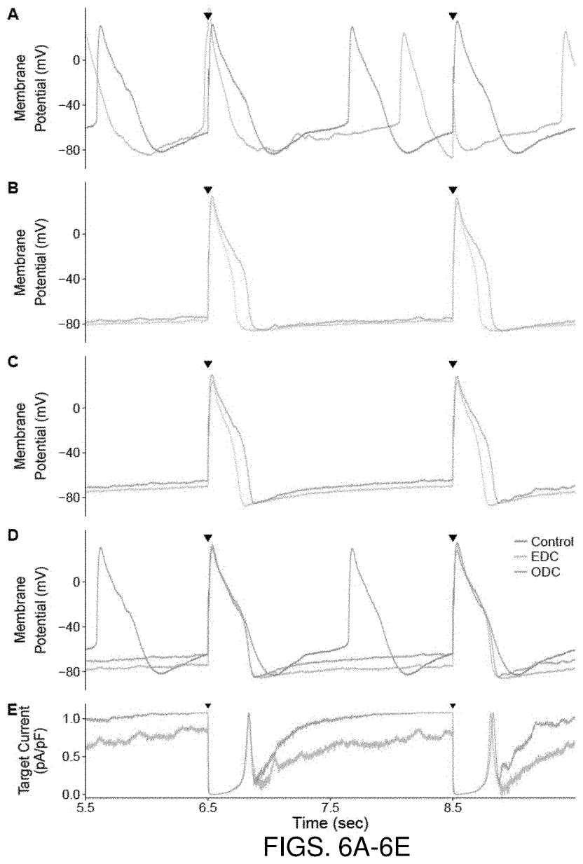

To investigate the feasibility of using the ODC platform for drug screening, it is important to determine if ODC can detect changes in AP morphology similar to EDC in the presence of an ion-channel modulator. The inventors used an I_{Kr} inhibitor, E-4031, because I_{Kr} is a dominant repolarizing current in iPSC-CMs and I_{Kr} inhibition assays are commonly used as drug toxicity assays. FIG. 6 shows an example after E-4031 treatment, demonstrating that the ODC platform mimics the effects of EDC on AP morphology. With E-4031 addition, the APD increases in the control, EDC and ODC conditions, as expected with I_{Kr} inhibition (FIG. 6A-C). Importantly, it is easier to observe the E-4031 induced AP prolongation under EDC and ODC compared to the control because there is less spontaneous activity (FIG. 6A-C). That said, like in FIG. 4, ODC does not maintain the membrane potential as well as EDC between APs, which can be seen in the different pre-stimulation potentials (and non-overlapping target currents) (FIG. 6D). However, the overlapping EDC and ODC stimulated APs and target currents during the AP indicate that ArchT achieved a similar effect as the electrode on AP morphology even if the same pre-stimulation potential was not achieved (FIGS. 6D, E). Thus, the ODC platform behaves like EDC in illuminating the expected changes on AP morphology and APD prolongation with E-4031 addition.

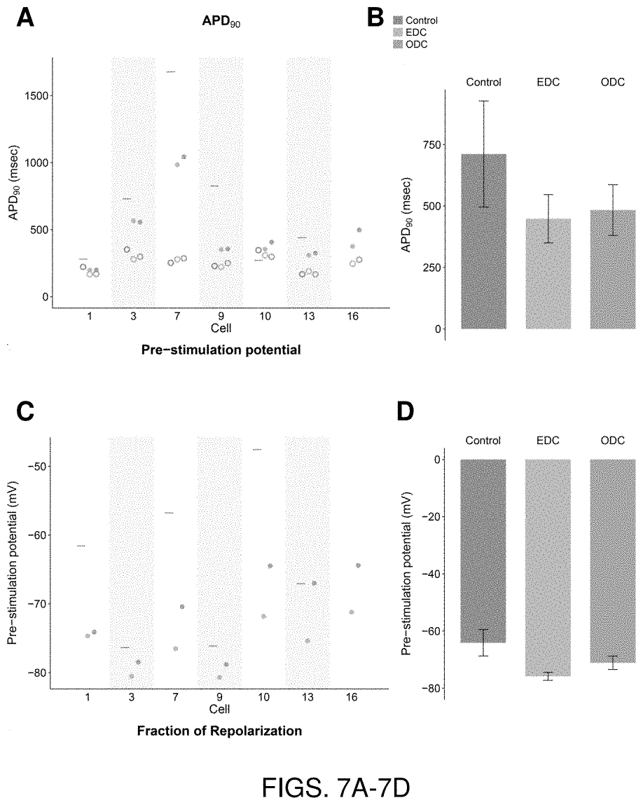

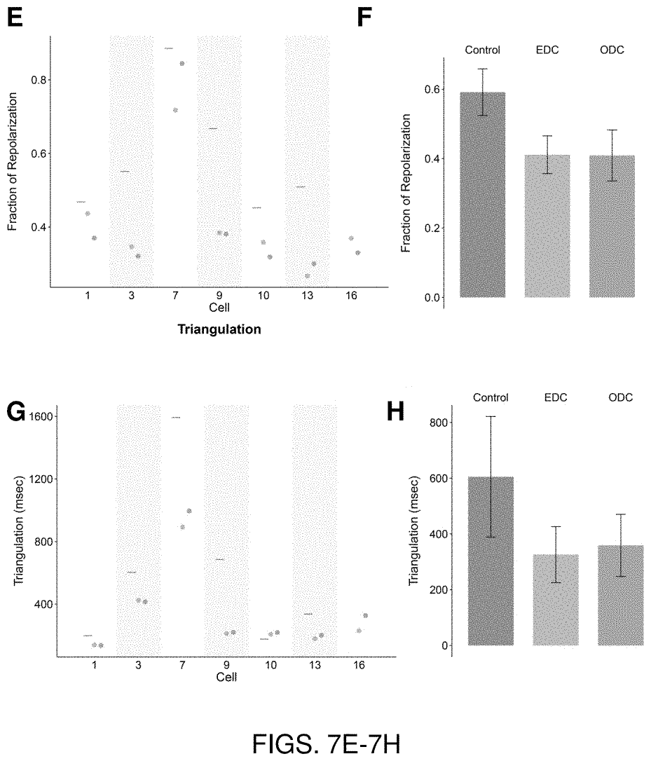

The effect of E-4031 with EDC and ODC across all cells is depicted in FIG. 7. In all cells, EDC and ODC were able to inhibit spontaneous activity seen in the absence of dynamic-clamp, allowing for an accurate measurement of AP characteristics. E-4031 increased the APD₉₀ under control, EDC and ODC conditions, as expected (FIG. 7A). Addition of simulated IK1 via both dynamic clamp platforms shortened the APD₉₀ compared to the control, as expected with increased repolarizing current. ODC also shortened the APD₉₀ to the same magnitude as EDC in individual cells and on average across all cells (FIGS. 7A, 7E). Cell 13 under the control condition yielded too much spontaneous activity after drug addition so that pacing did not override the intrinsic activity to yield stimulated action potentials. Similar to previous results without E-4031, the effect of ODC and EDC on pre-stimulation potential, fraction of repolarization time and triangulation compared to the control are similar (FIGS. 7B-7D). The average of characteristics across all cells confirm the overall AP morphology (APD90, fraction of repolarization, and triangulation) is nearly identical between EDC and ODC (FIG. 7H). Of note, with E-4031, the control (without dynamic clamp) cells exhibited more dramatic drug-induced effects in terms of APD prolongation and larger triangulation compared to EDC and ODC, which likely indicates that in lack of sufficient IK1, the hiPSC-CMs may be overly sensitive to classic hERG channel blockers. The agreement between the optical dynamic clamp and electrode dynamic clamp in the presence of E-4031 illustrates how ArchT can be used in place of an electrode in the context of drug screening.

Dynamic clamp is a technique that enables versatile and thorough probing of electrophysiology. However, its use for drug screening is limited because its standard implementation is low throughput. An optically-controlled version would enable more high-throughput applications. Here, inventors have disclosed proof-of-concept experiments, in which ArchT was controlled optically, injecting the I_{K1} target current and altering AP morphology similarly to electrode-based dynamic clamp.

### Example 5: Using Optical Dynamic Clamp for Drug Screening

The tedious nature of dynamic clamp restricts its use, but were it high throughput, it would open the possibility for its use during pre-clinical drug development. To increase throughput, an all-optical system would require an optical voltage readout, using, e.g., a voltage sensitive dye (VSD) or a genetically encoded voltage indicator (GEVI). The necessary requirements for compatibility in the ODC system would be defined by phototoxicity, brightness, responsiveness, and wavelength crosstalk. One of the biggest advantages of the ODC platform is that it is compatible with a variety of cell formats. In spatially-extended systems (e.g., large beating clusters and monolayers), the EDC platform is not applicable, while the ODC platform can be used to illuminate the arrhythmogenic effects of drugs. These more tissue-like formats capture “in-context” cell behavior, including electrotonic coupling and other chemical influences from neighboring cells, and therefore are preferred to single cells. Furthermore, all-optical methods enable high-precision space-time control in such multicellular systems, as illustrated recently in neurons (Sakai S, et al. Neurosci Res. 2013, 75(1):59-64) and in cardiac preparations (Burton R A B, et al. Nat Photonics. 2015, 9(12):813-6). This allows users to re-direct the control of electrical activity from the single-cell behavior to the emergent (wave) behavior (Burton R A B, et al. Nat Photonics. 2015, 9(12):813-6; Entcheva E, et al. J Physiol. 2016, 594(9):2503-10).

### Example 6: Versatility and Flexibility of ODC

With the right optogenetic tools and mathematical models, the optical dynamic clamp platform could open up more physiologically relevant formats for basic science research and drug development. Halorhodopsins, such as Natronomonas pharaonic halorhodopsin (NpHR) and its derivatives could also be used in this platform as an alternative to ArchT to inject a hyperpolarizing current given its fast kinetics (Mattis J, et al. Nat Methods. 2012, 9(2):159-72). Neither of these generate particularly high current, considering that they are light-sensitive ion pumps. GtACR1 is a Cl⁻ current with large amplitude (Govorunova E G, et al. Sci Rep, 2016, 6: 33530; Govorunova E G, et al. Science. 2015, 349(6248):647-50) that is also fast and can be used in ODC applications. BLINK1 is the first potassium-selective optogenetic tool available, but its kinetics are currently too slow for the near real-time feedback requirements of ODC (Cosentino C, et al. Science. 2015, 348(6235):707-10). There are also several depolarizing opsins available that can be used in conjunction with hyperpolarizing opsins, so that any inward or outward current can be represented in cardiomyocytes (Mattis J, et al. Nat Methods. 2012, 9(2):159-72; Ambrosi C M, et al. Prog Biophys Mol Biol. 2014, 115(2-3):294-304; Entcheva E. Am J Physiol—Heart Circ Physiol. 2013, 304(9):H1179-91). Optogenetic tools are being engineered to activate/deactivate faster, generate larger photocurrents, be permeable to specific ionic species or be activated by specific wavelengths. As these developments progress, users can choose which optogenetic tool best suits their needs in the ODC platform.

One known drawback of EDC is that an electrode can only electrically mimic a current but cannot account for endogenous secondary effects that affect electrophysiology, such as activation of exchangers, pumps, or Ca²⁺-dependent processes, which typically result from the change in intracellular ionic concentration. In this regard, because optogenetic tools alter the membrane potential by changing the intracellular ionic composition, the ODC platform may be more suitable for dynamic clamp than using an electrode because optogenetics can generate a custom-tailored current with the intended ionic species itself, reflecting how endogenous currents are generated. As with the standard dynamic clamp method, ArchT mimicked the electrical behavior of I_{K1}. But as potassium-selective tools are made compatible with the dynamic clamp system, the ODC platform may recapitulate both the electrical effect of I_{K1} and its effects from altering the intracellular potassium concentration. With the expansion of the optogenetic toolbox, the ODC platform will more accurately investigate true influence of an ionic current on electrophysiological behavior by generating the current with the relevant species.

Optogenetic tools are being creatively incorporated into automated high-throughput drug screening platforms (Dempsey G T, et al. J Pharmacol Toxicol Methods. 2016, 81:240-50; Klimas A, et al. Nat Commun. 2016, 7:ncomms11542; Clements I P, et al. Clin. and Transl. Neurophotonics. 2016, p. 96902C-96902C-10). This could also potentially expand on the use of automated multi-channel patch clamp systems to multicellular preparations. This hybrid system could continue to use a patch electrode to read the Vₘ but instead use optogenetic methods to inject a dynamic current into the multicellular format. The ODC method contributes a novel approach to probe electrical dynamics in iPSC-CMs and to better reveal how electrical activity is controlled. Here, an ODC application was demonstrated with ArchT to generate I_{K1} as target current in iPSC-CMs to simulate a more adult-like electrical phenotype. As the ODC platform develops, it should be possible to simulate abnormal currents or simulate heterogeneous current expression in iPSC-CM monolayers. This would provide a more powerful approach that enables researchers to address hypotheses that could not be investigated previously.

What is claimed is:

1. A kit comprising:
an excitable cell expressing at least one light-sensitive protein from an exogenous nucleic acid, wherein the light sensitive protein is selected from the group consisting of a light-sensitive ion channel and a light-sensitive ion pump; and
a computer readable media comprising instructions for performing an optical dynamic clamp on the cell, wherein the instructions comprise calculating a target ion current based on a measured membrane potential (Vₘ) using a predetermined relationship between a time-dependent Vₘ and an ion current; and calculating a target light intensity based on the target ion current, wherein exposing the excitable cell to the target light intensity results in an ion current from the light-sensitive protein that is equal to the target ion current.

2. The kit of claim 1, wherein the at least one light sensitive protein is selective for an ion selected from the group consisting of a potassium ion, a sodium ion, a chloride ion, a H⁺ ion and a calcium ion.

3. The kit of claim 1, wherein the excitable cell is selected from the group consisting of a neuron, a muscle cell and an excitable cell derived from an induced Pluripotent Stem Cell (iPSC).

4. The kit of claim 3, wherein the muscle cell is a cardiomyocyte.

5. The kit of claim 4, wherein the cardiomyocyte is an induced Pluripotent Stem Cell (iPSC)-derived cardiomyocyte.

6. The kit of claim 1, wherein the at least one light-sensitive protein is a channelrhodopsin, an anion-conducting channelrhodopsin, or a chimeric channelrhodopsin.

7. The kit of claim 6, wherein the channelrhodopsin is selected from the group consisting of Channelrhodopsin 1 (ChR1), Channelrhodopsin (ChR2), Volvox channelrhodopsin (VChR1), and Step function or bi-stable opsins (SFOs).

8. The kit of claim 1, wherein the light-sensitive protein is selected from a halorhodopsin (NpHR), an enhanced halorhodopsin eNpHR2.0, an enhanced halorhodopsin eNpHR3.0, an archaerhodopsin (Arch), the fungal opsin Mac and an enhanced bacteriorhodopsin (eBR).

9. The kit of claim 1, wherein the excitable cell expresses two different light-sensitive proteins, wherein each light sensitive protein is activated by a different wavelength of light.

10. The kit of claim 9, wherein one of the light sensitive proteins depolarizes the excitable cell when activated, and wherein the other light sensitive protein hyperpolarizes the excitable cell when activated.

11. A method for modulating the electrophysiology of a cell comprising:
(i) providing an excitable cell, an electrode, and a light source with a controllable light intensity or a controllable light wavelength, wherein the excitable cell expresses at least one light-sensitive protein from an exogenous nucleic acid, and wherein the light-sensitive protein is selected from a light-sensitive ion channel or a light-sensitive ion pump;
(ii) forming a high resistance electrical seal between the electrode and a membrane of the cell;
(iii) measuring the membrane potential (Vₘ) of the cell with the electrode;
(iv) calculating a target ion current based on the measured Vₘ using a predetermined relationship between a time-dependent Vₘ and an ion current; and
(v) adjusting the light intensity or the light wavelength of the light source thereby controlling an ion current from the light-sensitive protein until the measured ion current is equal to the target ion current.

12. The method of claim 11, further comprising repeating steps (iii) through (v).

13. The method of claim 11, wherein the calculating and adjusting steps are carried out by a computer.

14. The method of claim 11, wherein the predetermined relationship is determined from a control excitable cell.

15. The method of claim 11, wherein step (iv) further comprises calculating a target light intensity based on the target ion current.

16. The method of claim 11, wherein the at least one light-sensitive protein is selective for an ion selected from the group consisting of a potassium ion, a sodium ion, a chloride ion, a H⁺ ion and a calcium ion.

17. The method of claim 11, wherein the excitable cell is selected from the group consisting of a neuron, a muscle cell and an excitable cell derived from an induced Pluripotent Stem Cell (iPSC).

18. The method of claim 17, wherein the muscle cell is a cardiomyocyte.

19. The method of claim 18, wherein the cardiomyocyte is an induced Pluripotent Stem Cell (iPSC)-derived cardiomyocyte.

20. The method of claim 11, wherein the at least one light-sensitive protein is a channelrhodopsin, an anion-conducting channelrhodopsin, or a chimeric channelrhodopsin.

21. The method of claim 20, wherein the channelrhodopsin is selected from the group consisting of Channelrhodopsin 1 (ChR1), Channelrhodopsin (ChR2), Volvox channelrhodopsin 1 (VChR1), and Step function or bi-stable opsins (SFOs).

22. The method of claim 11, wherein the at least one light-sensitive protein is selected from a halorhodopsin (NpHR), an enhanced halorhodopsin eNpHR2.0, an enhanced halorhodopsin eNpHR3.0, an archaerhodopsin (Arch), the fungal opsin Mac and an enhanced bacteriorhodopsin (eBR).

23. The method of claim 11, wherein the excitable cell expresses at least two different light-sensitive proteins, wherein each light sensitive protein is activated by a different wavelength of light.

24. A method for modulating the electrophysiology of a cell comprising:
(i) providing an excitable cell, a light source with a controllable light intensity or light wavelength, and an optical detector, wherein the excitable cell expresses at least one light-sensitive protein from an exogenous nucleic acid, and wherein the light-sensitive protein is selected from a light-sensitive ion channel or a light-sensitive ion pump, and wherein the excitable cell further expresses an optogenetic sensor expressed from an exogenous nucleic acid, and wherein the optical detector produces a signal indicating a membrane potential (Vₘ);
(ii) measuring the Vₘ of the cell by measuring a signal from optogenic sensor with the optical detector;
(iii) calculating a target ion current based on the measured Vₘ using a predetermined relationship between a time-dependent Vₘ and an ion current;
(iv) adjusting the light intensity or the light wavelength of the light source thereby controlling an ion current from the light-sensitive protein until the measured ion current is equal to the target ion current.

25. The method of claim 24, further comprising repeating steps (ii) through (iv).

26. The method of claim 24, wherein the calculating and adjusting steps are carried out by a computer.

27. The method of claim 24, wherein the predetermined relationship is determined from a control excitable cell.

28. The method of claim 24, wherein step (iii) further comprises calculating a target light intensity based on the target ion current.

29. The method of claim 24, wherein the light-sensitive protein is selective for an ion selected from the group consisting of a potassium ion, a sodium ion, a chloride ion, a H⁺ ion and a calcium ion.

30. The method of claim 24, wherein the excitable cell is selected from the group consisting of a neuron, a muscle cell and an excitable cell derived from an induced Pluripotent Stem Cell (iPSC).

31. The method of claim 30, wherein the muscle cell is a cardiomyocyte.

32. The method of claim 31, wherein the cardiomyocyte is an induced Pluripotent Stem Cell (iPSC)-derived cardiomyocyte.

33. The method of claim 24, wherein the light-sensitive protein is a channelrhodopsin, an anion-conducting channelrhodopsin, or a chimeric channelrhodopsin.

34. The method of claim 33, wherein the channelrhodopsin is selected from the group consisting of Channelrhodopsin 1 (ChR1), Channelrhodopsin (ChR2), Volvox channelrhodopsin 1 (VChR1), and Step function or bi-stable opsins (SFOs).

35. The method of claim 24, wherein the light-sensitive protein is selected from a halorhodopsin (NpHR), an enhanced halorhodopsin eNpHR2.0, an enhanced halorhodopsin eNpHR3.0, an archaerhodopsin (Arch), the fungal opsin Mac and an enhanced bacteriorhodopsin (eBR).

36. The method of claim 24, wherein the optogenetic sensor is selected from the group consisting of arc lightning, D3cpVenus, G-CaMP and ASAP1.

37. The method of claim 11, wherein the excitable cell expresses two different light-sensitive proteins, wherein each light sensitive protein is activated by a different wavelength of light.
